(19) United States
(12) Patent Application Publication
(10) Pub. No.: US 2010/0021978 A1
(43) Pub. Date: Jan. 28, 2010

(54) METHODS AND ORGANISMS FOR PRODUCTION OF 3-HYDROXYPROPIONIC ACID

(75) Inventors: Mark J. Burk, San Diego, CA (US); Robin E. Osterhout, San Diego, CA (US)

Correspondence Address: MCDERMOTT, WILL & EMERY, 11682 EL CAMINO REAL, SUITE 400, SAN DIEGO, CA, 92130-2047, US

(73) Assignee: GENOMATICA, INC., San Diego, CA (US)

(21) Appl. No.: 12/508,547

(22) Filed: Jul. 23, 2009

Related U.S. Application Data
(60) Provisional application No. 61/083,123, filed on Jul. 23, 2008.
(60) Provisional application No. 61/118,366, filed on Nov. 26, 2008.
(60) Provisional application No. 61/119,319, filed on Dec. 2, 2008.

(51) Int. Cl. C12P 7/42; C12N 1/21

(52) U.S. Cl. 435/146; 435/252.3

(57) ABSTRACT

A non-naturally occurring microbial organism having a 3-hydroxypropanoic acid (3-HP) pathway includes at least one exogenous nucleic acid encoding 3-HP pathway enzyme expressed in a sufficient amount to produce 3-HP. The 3-HP pathway includes a 2-keto acid decarboxylase, a CoA-dependent oxaloacetate dehydrogenase, or a malate decarboxylase. A method for producing 3-HP includes culturing a non-naturally occurring microbial organism having a 3-HP pathway that includes at least one exogenous nucleic acid encoding a 3-HP pathway enzyme expressed in a sufficient amount to produce 3-HP under conditions and for a sufficient period of time to produce 3-HP. The 3-HP pathway includes a 2-keto acid decarboxylase, a CoA-dependent oxaloacetate dehydrogenase, or a malate decarboxylase.

This application claims the benefit of priority of U.S. Provisional application Ser. No. 61/083,123, filed Jul. 23, 2008; U.S. Provisional application Ser. No. 61/118,366, filed Nov. 26, 2008; and U.S. Provisional application Ser. No. 61/119,319, filed Dec. 2, 2008, each of which the entire contents is incorporated herein by reference.

### BACKGROUND OF THE INVENTION

This invention relates generally to the production of commodity and specialty chemicals and, more specifically to an integrated bioprocess for producing 3-hydroxypropionic acid (3-HP).

The compound 3-hydroxypropionic acid (3-hydroxypropionate or 3-HP) is a three-carbon carboxylic acid that has industrial potential as a building block for a number of commodity and specialty chemicals. Compounds that can be produced from 3-HP by chemical synthesis include polymer precursors such as 3-HP, acrylamide, methyl 3-HP, and 1,3-propanediol; chemical intermediates such as malonic acid, and alcohol esters of 3-HP. 3-HP itself also is used in the nutritional industry as a food preservative. Although the above compounds can be produced from petroleum feedstocks, the ability to produce the entire family of 3-HP derived products from a platform chemical, preferably made from renewable resources, would be useful.

Several chemical synthesis routes have been described to produce 3-HP, and biocatalytic routes have also been disclosed (WO 01/16346 to Suthers et al.). However, chemical synthesis of 3-HP is costly and inefficient.

Despite the efforts and reports purporting the development of biocatalytic routes for the production of 3-HP, the approaches employed have several drawbacks which hinder applicability in commercial settings. The strains produced by these methods can be unstable in commercial fermentation processes due to selective pressures favoring the unaltered or wild-type parental counterparts. Thus, there exists a need for microorganisms having commercially beneficial characteristics that can efficiently produce commercial quantities of 3-HP. The present invention satisfies this need and provides related advantages as well.

### SUMMARY OF THE INVENTION

In some aspects, the present invention relates to a non-naturally occurring microbial organism having a 3-hydroxypropanoic acid (3-HP) pathway that includes at least one exogenous nucleic acid encoding 3-HP pathway enzyme expressed in a sufficient amount to produce 3-HP. The 3-HP pathway includes an 2-keto acid decarboxylase.

In other aspects, the present invention relates to a method for producing 3-HP that induces culturing a non-naturally occurring microbial organism having a 3-HP pathway. The pathway includes at least one exogenous nucleic acid encoding a 3-HP pathway enzyme expressed in a sufficient amount to produce 3-HP under conditions and for a sufficient period of time to produce 3-HP. The 3-HP pathway includes an 2-keto acid decarboxylase.

In yet other aspects, the present invention relates to a non-naturally occurring microbial organism having a 3-hydroxypropanoic acid (3-HP) pathway that includes at least one exogenous nucleic acid encoding a 3-HP pathway enzyme expressed in a sufficient amount to produce 3-HP. The 3-HP pathway includes a CoA-dependent oxaloacetate dehydrogenase.

In yet other aspects, the present invention provides a method for producing 3-HP that includes culturing a non-naturally occurring microbial organism having a malonyl-CoA to 3-HP pathway. The pathway includes at least one exogenous nucleic acid encoding a malonyl-CoA to 3-HP pathway enzyme expressed in a sufficient amount to produce 3-HP under conditions and for a sufficient period of time to produce 3-HP. The malonyl-CoA to 3-HP pathway includes a CoA-dependent oxaloacetate dehydrogenase.

In yet still further aspects, the present invention relates to a non-naturally occurring microbial organism having a 3-hydroxypropanoic acid (3-HP) pathway that includes at least one exogenous nucleic acid encoding a 3-HP pathway enzyme expressed in a sufficient amount to produce 3-HP. The 3-HP pathway includes a malate decarboxylase.

Finally, in some aspects, the present invention provides a method for producing 3-HP that includes culturing a non-naturally occurring microbial organism having a malonyl-CoA to 3-HP pathway. The pathway includes at least one exogenous nucleic acid encoding a malate to 3-HP pathway enzyme expressed in a sufficient amount to produce 3-HP under conditions and for a sufficient period of time to produce 3-HP. The malonyl-CoA to 3-HP pathway includes a malate decarboxylase.

### BRIEF DESCRIPTION OF THE DRAWINGS

FIG. 1 shows a biosynthetic pathway for the production of 3-HP having a decarboxylase for the conversion of oxaloacetate to malonate semialdehyde.

FIG. 2 shows a biosynthetic pathway for the production of 3-HP having a dehydrogenase for the conversion of oxaloacetate to malonyl-CoA.

FIG. 3 shows a comparison of enzyme candidates for catalyzing the conversion of oxaloacetate to malonyl-CoA. Pathways encoded by candidate 2-keto-acid dehydrogenase complexes: A. shows the transformation in FIG. 2 (step 2), B. conversion of alpha-ketoglutarate to succinyl-CoA, catalyzed by alpha-ketoglutarate dehydrogenase complex and branched-chain ketoacid dehydrogenase complex in some organisms, C. conversion of 2-oxobutanoate to propanoyl-CoA, catalyzed by branched-chain ketoacid dehydrogenase complex and the pyruvate dehydrogenase complex in some organisms, and D. conversion of pyruvate to acetyl-CoA by the pyruvate dehydrogenase complex.

FIG. 4 shows a biosynthetic pathway for the production of 3-HP having a malate decarboxylase for the conversion of malate to 3-HP.

### DETAILED DESCRIPTION OF THE INVENTION

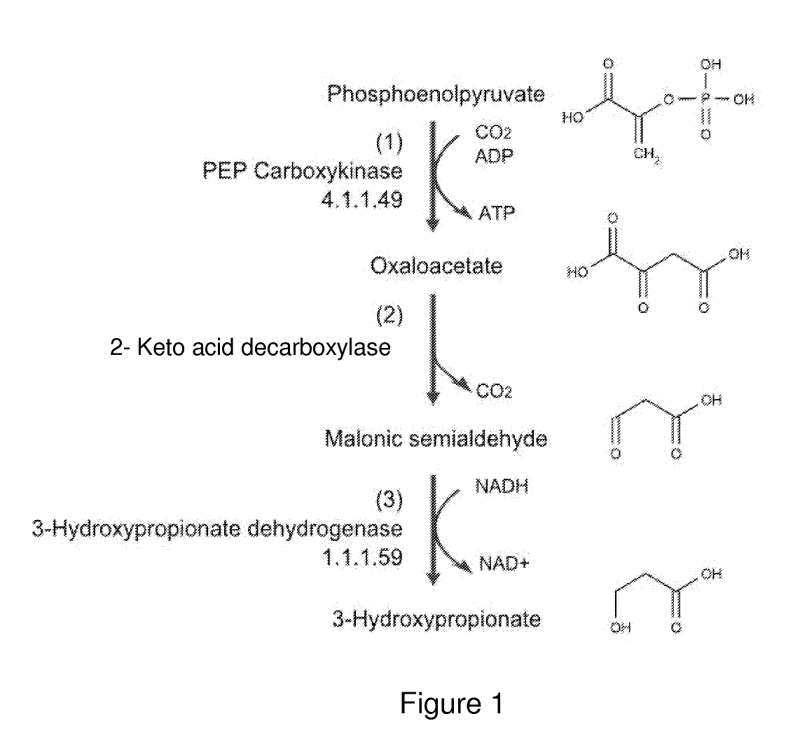

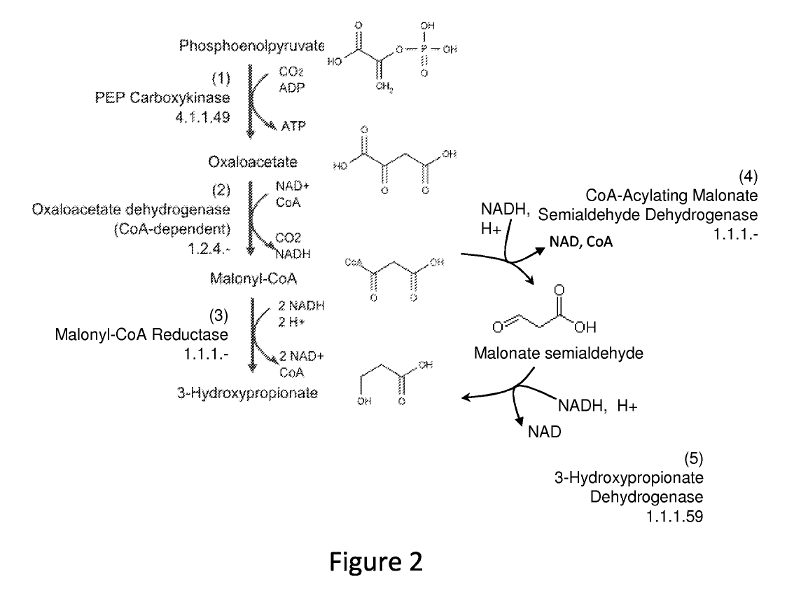

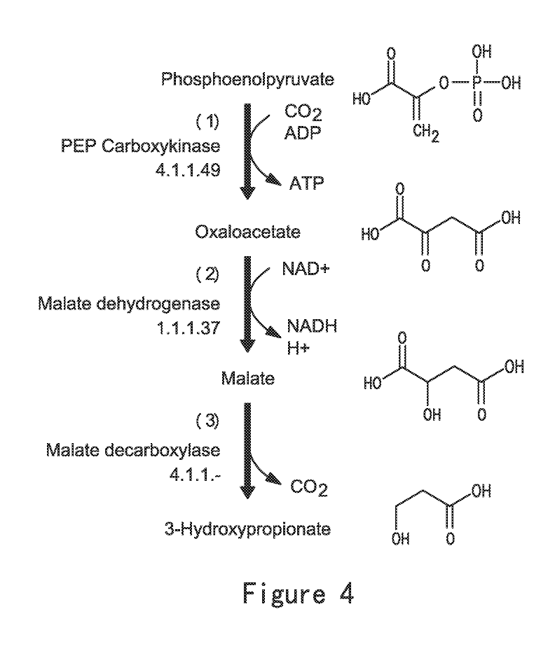

This invention is directed, in part, to the design and production of cells and organisms incorporating biosynthetic pathways for the production of 3-hydroxypropionic acid (3-HP). In particular, this invention is directed to methods of producing 3-HP involving primary metabolic production of oxaloacetate, followed by decarboxylation and subsequent reduction of resultant malonate semialdehyde as shown in FIG. 1. Alternatively, 3-HP production involves a pathway that includes a CoA-dependent dehydrogenation followed by the two-step reduction of the resultant malonyl-CoA as shown in FIG. 2. In yet another alternative, 3-HP production involves a pathway that involves the decarboxylation of malate as shown in FIG. 4.

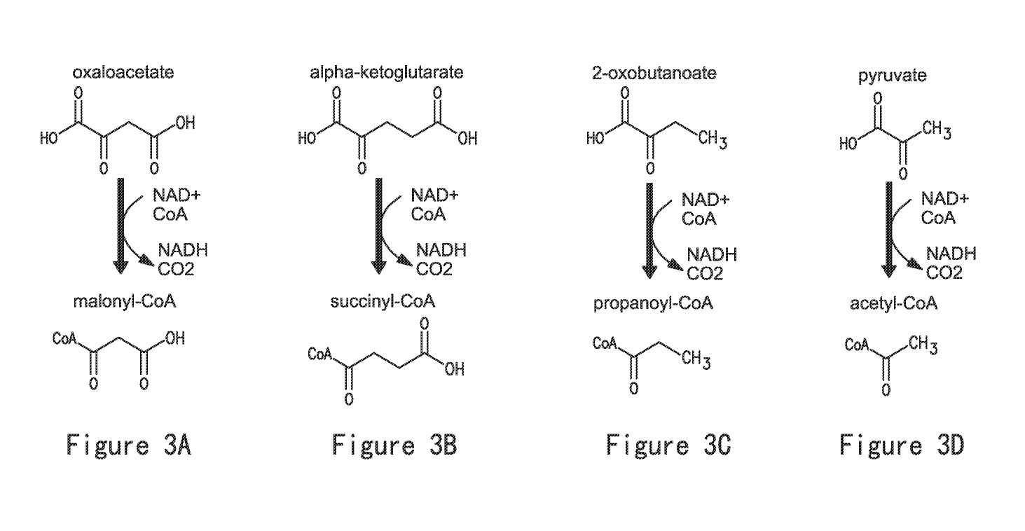

Oxaloacetate is a common intermediate of central cellular metabolism and is produced by the tricarboxylic acid cycle (TCA) cycle. Central metabolites are good targets for metabolic engineering as they are often constitutively produced during basal metabolism. In addition to the production of oxaloacetate by way of carboxylation of phosphoenolpyruvate (PEP) as shown in FIGS. 1 and 2, oxaloacetate is biosynthetically accessible from malate in the TCA cycle by oxidation mediated by malate dehydrogenase. Furthermore, oxaloacetate can be generated from citrate by, for example, action of an ATP citrate lyase in the presence of ATP and coenzyme A. FIG. 1 shows a novel process to 3-HP which involves treatment of oxaloacetate with a decarboxylase enzyme in a pathway leading to 3-HP via malonate semialdehyde. The final reduction step can be carried out by a dehydrogenase such as 3-hydroxypropionate dehydrogenase in the presence of NADH or NADPH or by a reductase. FIG. 2 shows a biosynthetic scheme to 3-HP which involves treatment of oxaloacetate with a CoA-dependent dehydrogenase enzyme in a pathway leading to 3-HP via malonyl-CoA. The final reduction steps can be carried out by a single enzyme possessing alcohol and aldehyde dehydrogenase functionalities, or a pair of enzymes possessing these functions. As shown in FIG. 3, oxaloacetate derived from phosphoenolpyruvate can also be converted to malate by the TCA cycle enzyme malate dehydrogenase. Malate can subsequently be decarboxylated to form 3-HP.

Many different substrates derived from renewable feedstocks, such as glucose, xylose, arabinose, sorbitol, sucrose, glycerol, or even synthesis gas (a mixture of carbon monoxide, hydrogen and carbon dioxide), can serve as carbon and energy sources for a fermentation process. Each of these substrates can be used for biological production of 3-HP.

As used herein, the term “non-naturally occurring” when used in reference to a microorganism of the invention is intended to mean that the microorganism has at least one genetic alteration not normally found in a wild-type strain of the referenced species. The genetic alteration can be a gene deletion or some other functional disruption of the genetic material.

As used herein the term “parent decarboxylase” refers to both wild-type and previously engineered decarboxylases that serve as a starting point for further optimization of the decarboxylation activity. Optimizations can include not only changes made to the nucleic acid sequence encoding the decarboxylase, but also post-translational modifications to the enzyme product.

As used herein, the term “isolated” when used in reference to a microbial organism is intended to mean an organism that is substantially free of at least one component as the referenced microbial organism is found in nature. The term includes a microbial organism that is removed from some or all components as it is found in its natural environment. The term also includes a microbial organism that is removed from some or all components as the microbial organism is found in non-naturally occurring environments. Therefore, an isolated microbial organism is partly or completely separated from other substances as it is found in nature or as it is grown, stored or subsisted in non-naturally occurring environments. Specific examples of isolated microbial organisms include partially pure microbes, substantially pure microbes and microbes cultured in a medium that is non-naturally occurring.

Furthermore, “isolated” can be used in reference to a substantially purified protein, enzyme, or the like, or a nucleic acid sequence that can be subsequently inserted into a host microbial organism by standard methods known in the art.

As used herein, the terms “microbial,” “microbial organism” or “microorganism” are intended to mean a prokaryotic or eukaryotic cell or organism having a microscopic size. The terms are intended to include bacteria of all species and eukaryotic organisms such as yeast and fungi. The terms also include cell cultures of any species that can be cultured for the production of a biochemical.

As used herein, the term “3-hydroxypropionic acid” or “3-HP” is intended to mean the carboxylic acid C₃H₆O₃ having a molecular mass of 90.08 g/mol and a pKa of 4.5. It also is known in the art as hydr3-HP and ethylene lactic acid. The term “3-hydroxypropionic acid” as it is used herein is intended to include any of its various 3-hydroxypropionate salt forms. Chemically, 3-hydroxyproprionate corresponds to a salt or ester of 3-hydroxypropionic acid. Therefore, 3-hydroxypropionic acid and 3-hydroxypropionate refer to the same compound, which can be present in either of the two forms depending on the pH of the solution. Therefore, the terms 3-hydroxypropionic acid, 3-hydroxypropionate and 3-HP as well as its other art recognized names hydr3-HP and ethylene lactic acid are used synonymously herein.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid of the invention can utilize either or both a heterologous or homologous encoding nucleic acid.

As used herein, the term “metabolic modification” is intended to refer to a biochemical reaction that is altered from its naturally occurring state. Metabolic modifications can include, for example, elimination of a biochemical reaction activity by functional disruptions of one or more genes encoding an enzyme participating in the reaction. Metabolic modifications can also include enhancements of a biochemical reaction activity by, for example, insertion of extra copies of genes encoding an enzyme participating in the reaction.

As used herein, the term “stable” when used in reference to production of a biochemical product is intended to refer to microorganism that can be cultured for greater than five generations without loss of the coupling between growth and biochemical synthesis. Generally, stable growth-coupled biochemical production will be greater than 10 generations, particularly stable growth-coupled biochemical production will be greater than about 25 generations, and more particularly, stable growth-coupled biochemical production will be greater than 50 generations, including indefinitely. Stable growth-coupled production of a biochemical can be achieved, for example, by deletion of a gene encoding an enzyme catalyzing each reaction within a set of metabolic modifications. The stability of growth-coupled production of a biochemical can be enhanced through multiple deletions, significantly reducing the likelihood of multiple compensatory reversions occurring for each disrupted activity.

Those skilled in the art will understand that the metabolic modifications exemplified herein are described with reference to E. coli genes and their corresponding metabolic reactions. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous gene insertions or gene disruptions in the other species.

As used herein, an “ortholog” is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less that 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the growth-coupled production of a biochemical product, those skilled in the art will understand that the orthologous gene harboring the metabolic activity to be disrupted is to be chosen for construction of the non-naturally occurring microorganism. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, “paralogs” are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A “nonorthologous gene displacement” is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene compared to a gene encoding the function sought to be substituted. Therefore, a nonorthologous gene includes, for example, a paralog or an unrelated gene.

Therefore, in identifying and constructing the non-naturally occurring microorganisms of the invention for production of a biochemical, those skilled in the art will understand that applying the teaching and guidance provided herein to a particular species that the identification of metabolic modifications should include identification and disruption of orthologs. To the extent that paralogs and/or nonorthologous gene displacements are present in the referenced microorganism that encode an enzyme catalyzing a similar or substantially similar metabolic reaction, those skilled in the art also can eliminate these evolutionally related genes to ensure that any functional redundancy in enzymatic activities do not short circuit the designed metabolic modifications.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compared and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarly to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: -2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

The invention provides a method of producing a non-naturally occurring microorganism having a biosynthetic pathway for the production of 3-hydroxypropionic acid. As will become evident, the teachings contained herein will enable the development of methods for decarboxylating oxaloacetate through the use of naturally occurring or altered decarboxylases. Such alterations can be introduced through a variety of directed and/or adaptive evolution methods.

In some embodiments, the present invention provides a non-naturally occurring microbial organism having a 3-HP pathway that includes at least one exogenous nucleic acid encoding a 3-HP pathway enzyme expressed in a sufficient amount to produce 3-HP. One exemplary exogenous nucleic acid in the 3-HP pathway is a decarboxylase capable of decarboxylating oxaloacetate to malonate semialdehyde. In some embodiments, this exogenous nucleic acid is a heterologous nucleic acid. Preferably, the microbial organism is cultured in a substantially anaerobic culture medium.

As shown in FIG. 1, in the second biosynthetic step oxaloacetate is decarboxylated to form malonic semialdehyde by an oxaloacetate decarboxylase. Decarboxylases (also known as carboxy lyases) catalyze the loss of carbon dioxide from an organic compound or a cellular metabolite possessing a carboxylic acid function. Decarboxylases are prevalent in nature and can require either pyridoxal phosphate, thiamine pyrophosphate, or pyruvate as a co-factor, although many require no bound co-factors. Over 50 decarboxylase enzymes have been reported and characterized by biochemical and/or analytical methods.

The native oxaloacetate decarboxylase in E. coli (EC 4.1.1.3), encoded by gene eda, acts on the terminal acid of oxaloacetate to form pyruvate. Because this reaction can compete with decarboxylation at the keto-acid position, this gene can be knocked out in a host strain for producing 3-HP using this pathway. Enzymes useful for this step include, for example, pyruvate decarboxylase (EC 4.1.1.1), benzoylformate decarboxylase (4.1.1.7), alpha-ketoglutarate decarboxylase (EC 4.1.1.71), branched-chain alpha-keto-acid decarboxylase (4.1.1.72), and indolepyruvate decarboxylase (EC 4.1.1.74). These classes of decarboxylases are CoA and NADH-independent, they utilize thiamine diphosphate as a cofactor, and the interaction of the substrate with the enzyme-bound cofactor is thought to be the rate-limiting step for enzyme activation (Hubner, G., R. Weidhase, and A. Schellenberger. 1978. The mechanism of substrate activation of pyruvate decarboxylase: a first approach. Eur. J Biochem. 92:175-181.). Pyruvate decarboxylase and benzoylformate decarboxylase have broad substrate ranges for diverse keto-acids and have been characterized in structural detail. Another exemplary class of decarboxylases that can be used to decarboxylate oxaloacetate is alpha-ketoglutarate decarboxylases (AKG-decarboxylases) which decarboxylate the homologous substrate alpha-ketoglutaric acid as shown below in Scheme I.

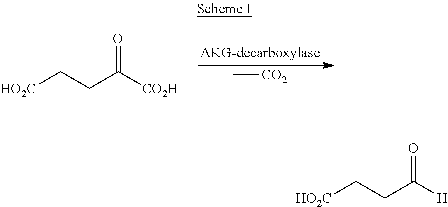

Pyruvate decarboxylase (PDC), also termed keto-acid decarboxylase, is a key enzyme in alcoholic fermentation, catalyzing the decarboxylation of pyruvate to acetaldehyde. This enzyme has a broad substrate range for aliphatic 2-keto acids including 2-ketobutyrate, 2-ketovalerate, 3-hydroxypyruvate and 2-phenylpyruvate (Li and Jordan, Biochemistry 38:10004-10012 (1999)). The PDC from Zymomonas mobilus, encoded by pdc, has been a subject of directed engineering studies that altered the affinity for different substrates (Siegert et al., Protein Eng Des Sel 18:345-357 (2005)). The PDC from Saccharomyces cerevisiae has also been extensively studied, engineered for altered activity, and functionally expressed in E. coli (Killenberg-Jabs et al., Eur. J. Biochem. 268:1698-1704 (2001); Li and Jordan Biochemistry 38:10004-10012 (1999); ter Schure et al., Appl. Environ. Microbiol. 64:1303-1307 (1998)). The crystal structure of this enzyme is available (Killenberg-Jabs et al., Eur. J. Biochem. 268:1698-1704 (2001)). Other well-characterized PDC candidates include the enzymes from Acetobacter pasteurians (Chandra et al., Arch. Microbiol. 176:443-451 (2001)) and Kluyveromyces lactis (Krieger et al. Eur. J. Biochem. 269:3256-3263 (2002)). Table 1 below summarizes gene information for various pyruvate decarboxylases.

TABLE 1

| | GenBank | | |
|---|---|---|---|
| Gene | ID | GI | Organism |
| pdc | P06672.1 | 118391 | Zymomonas mobilus |
| pdc1 | P06169 | 30923172 | Saccharomyces cerevisiae |
| pdc | Q8L388 | 75401616 | Acetobacter pasteurians |
| pdc1 | Q12629 | 52788279 | Kluyveromyces lactis |

Like PDC, benzoylformate decarboxylase has a broad substrate range and has been the target of enzyme engineering studies. The enzyme from Pseudomonas putida has been extensively studied and crystal structures of this enzyme are available (Hasson et al., Biochemistry 37:9918-9930 (1998); Polovnikova et al., Biochemistry 42:1820-1830 (2003).). Site-directed mutagenesis of two residues in the active site of the Pseudomonas putida enzyme altered the affinity (Km) of naturally and non-naturally occuring substrates (Siegert et al., Protein Eng Des Sel 18:345-357 (2005)). The properties of this enzyme have been further modified by directed engineering (Lingen. et al. Protein Eng 15:585-593 (2002); Lingen et al., Chembiochem. 4:721-726 (2003)). The enzyme from Pseudomonas aeruginosa, encoded by mdlC, has also been characterized experimentally (Barrowman et al., FEMS Microbiology Letters 34:57-60 (1986)). Additional gene candidates from Pseudomonas stutzeri, Pseudomonas fluorescens and other organisms can be inferred by sequence homology or identified using a growth selection system developed in Pseudomonas putida (Henning et al., Appl. Environ. Microbiol. 72:7510-7517 (2006)). Table 2 below summarizes gene information for various benzoylformate decarboxylases.

TABLE 2

| Gene | GenBank ID | GI | Organism |
|---|---|---|---|
| mdlC | P20906.2 | 3915757 | Pseudomonas putida |
| mdlC | Q9HUR2.1 | 81539678 | Pseudomonas aeruginosa |
| dpgB | ABN80423.1 | 126202187 | Pseudomonas stutzeri |
| ilvB-1 | YP_260581.1 | 70730840 | Pseudomonas fluorescens |

A third enzyme capable of decarboxylating 2-oxoacids is alpha-ketoglutarate decarboxylase (KGD). The substrate range of this class of enzymes has not been studied extensively to date. The KDC from Mycobacterium tuberculosis (Tian et al., Proc Natl Acad Sci US.A. 102:10670-10675 (2005)) has been cloned and functionally expressed in other internal projects by Applicants. However, it is not an ideal candidate for strain engineering because it is large (˜130 kD) and GC-rich. KDC enzyme activity has been detected in several species of rhizobia including Bradyrhizobium japonicum and Mesorhizobium loti (Green et al., J. Bacteriol. 182:2838-2844 (2000)). Although the KDC-encoding gene(s) have not been isolated in these organisms, the genome sequences are available and several genes in each genome are annotated as putative KDCs. A KDC from Euglena gracilis has also been characterized but the gene associated with this activity has not been identified to date (Shigeoka and Nakano, Arch. Biochem. Biophys. 288:22-28 (1991)), although the first twenty amino acids starting from the N-terminus were sequenced MTYKAPVKDVKFLLDKVFKV. The gene could be identified by testing candidate genes containing this N-terminal sequence for KDC activity. Table 3 below summarizes gene information for various alpha-ketoglutarate decarboxylases.

TABLE 3

| Gene | GenBank ID | GI | Organism |
|---|---|---|---|
| kgd | O50463.4 | 160395583 | Mycobacterium tuberculosis |
| kgd | NP_767092.1 | 27375563 | Bradyrhizobium japonicum |
| | | | USDA110 |
| kgd | NP_105204.1 | 13473636 | Mesorhizobium loti |

A fourth enzyme that can catalyze this step is branched chain alpha-ketoacid decarboxylase (BCKA). This class of enzyme has been shown to act on a variety of compounds varying in chain length from 3 to 6 carbons (Oku and Kaneda, J Biol Chem. 263:18386-18396 (1988); Smit et al., 2005 Appl Environ Microbiol 71:303-311 (2005)). The enzyme in Lactococcus lactis has been characterized on a variety of branched and linear substrates including 2-oxobutanoate, 2-oxohexanoate, 2-oxopentanoate, 3-methyl-2-oxobutanoate, 4-methyl-2-oxobutanoate and isocaproate (Smit et al., Appl Environ Microbiol 71:303-311 (2005)). The enzyme has been structurally characterized (Berthold et al., Acta Crystallogr. D Biol Crystallogr. 63:1217-1224 (2007)). Sequence alignments between the Lactococcus lactis enzyme and the pyruvate decarboxylase of Zymomonas mobilus indicate that the catalytic and substrate recognition residues are nearly identical (Siegert et al., Protein Eng Des Sel 18:345-357 (2005)), so this enzyme would be a promising candidate for directed engineering. Decarboxylation of alpha-ketoglutarate by a BCKA was detected in Bacillus subtilus; however, this activity was low (5%) relative to activity on other branched-chain substrates (Oku and Kaneda et al., J Biol Chem. 263:18386-18396 (1988)) and the gene encoding this enzyme has not been identified to date. Additional BCKA gene candidates can be identified by homology to the Lactococcus lactis protein sequence. Many of the high-scoring BLASTp hits to this enzyme are annotated as indolepyruvate decarboxylases (EC 4.1.1.74). Indolepyruvate decarboxylase (IPDA) is an enzyme that catalyzes the decarboxylation of indolepyruvate to indoleacetaldehyde in plants and plant bacteria. Table 4 below shows the gene information for branched chain alpha-ketoacid decarboxylase (BCKA).

TABLE 4

| Gene | GenBank ID | GI | Organism |
|---|---|---|---|
| kdcA | AAS49166.1 | 44921617 | Lactococcus lactis |

Recombinant branched chain alpha-keto acid decarboxylase enzymes derived from the E1 subunits of the mitochondrial branched-chain keto acid dehydrogenase complex from Homo sapiens and Bos taurus have been cloned and functionally expressed in E. coli (Davie et al., J. Biol. Chem. 267:16601-16606 (1992); Wynn et al. J. Biol. Chem. 267:1881-1887 (1992); Wynn et al., J. Biol. Chem. 267:12400-12403 (1992)). In these studies, the it was found that co-expression of chaperonins GroEL and GroES enhanced the specific activity of the decarboxylase by 500-fold (Wynn et al., J. Biol. Chem. 267:12400-12403 (1992)). These enzymes are composed of two alpha and two beta subunits. Table 5 below shows the gene information for recombinant branched chain alpha-ketoacid decarboxylase (BCKA).

TABLE 5

| Gene | GenBank ID | GI | Organism |
|---|---|---|---|
| BCKDHB | NP_898871.1 | 34101272 | Homo sapiens |
| BCKDHA | NP_000700.1 | 11386135 | Homo sapiens |
| BCKDHB | P21839 | 115502434 | Bos taurus |
| BCKDHA | P11178 | 129030 | Bos Taurus |

If the desired activity or productivity of a given enzyme is less than optimal in the conversion of oxaloacetate to malonate semialdehyde, or if malonate semialdehyde production inhibits the decarboxylase enzymes, the decarboxylase enzymes can be evolved using known protein engineering methods to achieve the required performance. Importantly, it was shown through the use of chimeric enzymes that the C-terminal region of decarboxylases appears to be responsible for substrate specificity (Barthelmebs et al., Appl. Environ. Microbiol. 67:1063-1069 (2001)). Accordingly, directed evolution experiments to broaden the specificity of decarboxylases in order to gain activity with oxaloacetate can be focused on the C-terminal region of these enzymes.

In some embodiments, the non-naturally occurring microbial organism includes an exogenous nucleic acid encoding a phosphoenolpyruvate carboxykinase. The first step shown in FIG. 1 is the conversion of phosphoenolpyruvate to oxaloacetate by phosphoenolpyruvate carboxykinase (PPCK). This conversion is catalyzed by the enzyme PEP carboxylase in wild type E. coli during growth on carbohydrates. However, this enzyme results in a net decrease of energy available for biosynthetic pathways because the high energy phosphate bond contained in each PEP molecule is lost as inorganic phosphate upon conversion to oxaloacetate. This energetic limitation can be remedied by introducing a reversible phosphoenolpyruvate carboxykinase (PPCK) enzyme, which unlike PEP carboxylase, can generate one ATP per phosphoenolpyruvate molecule converted to oxaloacetate.

PPCK is known to produce oxaloacetate from PEP in rumen bacteria such as Mannheimia succiniciproducens (Hong et al., Nat Biotechnol 22:1275-81 (2004)). However, the role of this enzyme, encoded by pck, in producing oxaloacetate in E. coli, is thought to be minor as compared to PEP carboxylase possibly due to the higher Kₘ for bicarbonate of PPCK (Kim et al., Appl Environ Microbiol 70:1238-1241 (2004)). Nevertheless, activity of the native E. coli PEP carboxykinase from PEP towards oxaloacetate has been recently demonstrated in ppc mutants of E. coli K-12 (Kwon et al., Journal of Microbiology and Biotechnology 16:1448-1452 (2006)). These strains exhibited no growth defects and had increased succinate production at high NaHCO₃ concentrations.

Examples of non-native PEP carboxykinase genes that have been cloned into E. coli include those from M. succiniciproducens (Lee et al., Biotechnol. Bioprocess Eng. 7:95-99 (2002)), Anaerobiospirillum succiniciproducens (Laivenieks et al., Appl Environ Microbiol 63:2273-2280 (1997)), and Actinobacillus succinogenes (Kim et al., Appl Environ Microbiol 70:1238-1241 (2004)). Applicants also disclose herein their finding that the PPCK enzyme encoded by Haemophilus influenza is highly efficient. Table 6 below summarizes the relevant PEP carboxykinase gene information.

TABLE 6

| Gene | GenBank ID | GI | Organism |
|---|---|---|---|
| pck | NP_417862.1 | 16131280 | Escherichia coli K12 strain |
| | | | MG1655 |
| pckA | YP_089485.1 | 52426348 | Mannheimia succiniciproducens |
| pckA | O09460.1 | 3122621 | Anaerobiospirillum |
| | | | succiniciproducens |
| pckA | Q6W6X5 | 75440571 | Actinobacillus succinogenes |
| pckA | P43923.1 | 1172573 | Haemophilus influenza |

In still further embodiments, the non-naturally occurring microbial organism includes an exogenous nucleic acid encoding a 3-hydroxypropionate dehydrogenase, which when run in a reverse direction in the presence of NADH or NADPH, is capable of reducing malonate semialdehyde to 3-HP. In alternate embodiments the exogenous nucleic acid for carrying out the reduction of malonate semialdehyde is a reductase. Three enzymes are known to catalyze the conversion of malonate semialdehyde to 3-HP: NADH-dependent 3-hydroxy-propionate dehydrogenase, NADPH-dependent malonate semialdehyde reductase, and NADH-dependent 3-hydroxyisobutyrate dehydrogenases. NADH-dependent 3-hydroxy-propionate dehydrogenase participates in beta-alanine biosynthesis pathways from propionate in bacteria and plants. This enzyme has not been associated with a gene in any organism to date. NADPH-dependent malonate semialdehyde reductase catalyzes the reverse reaction in autotrophic CO2-fixing bacteria. Although the enzyme activity has been studied in Metallosphaera sedula, the identity of the gene is not known.

Several 3-hydroxyisobutyrate dehydrogenase enzymes have also been shown to convert malonic semialdehyde to 3-HP. Three genes exhibiting this activity are mmsB from Pseudomonas aeruginosa PAO1 (Gokarn et al., U.S. Pat. No. 7,393,676), mmsB from Pseudomonas putida KT2440 (Liao et al., U.S. Patent Application No. 20050221466), and mmsB from Pseudomonas putida E23 (Chowdhury et al., Biosci. Biotechnol. Biochem. 60:2043-2047 (1996)). The protein from Pseudomonas putida E23 has been characterized and functionally expressed in E. coli; however, its activity on 3-HP was relatively low (Chowdhury et al., Biosci. Biotechnol. Biochem. 60:2043-2047 (1996)). An enzyme with 3-hydroxybutyrate dehydrogenase activity in Alcaligenes faecalis M3A has also been identified (Gokarn et al., U.S. Pat. No. 7,393,676; Liao et al., U.S. Patent Application No. 20050221466). Enzymes with 3-hydroxybutyrate dehydrogenase activity in Alcaligenes faecalis M3A and Rhodobacter spaeroides have also been identified. Table 7 below summarizes the relevant gene information.

TABLE 7

| Gene | GenBank ID | GI | Organism |
|---|---|---|---|
| mmsB | NP_252259.1 | 15598765 | Pseudomonas aeruginosa |
| | | | PAO1 |
| mmsB | NP_746775.1 | 26991350 | Pseudomonas putida KT2440 |
| mmsB | JC7926 | 60729613 | Pseudomonas putida E23 |
| orfB1 | AAL26884 | 16588720 | Rhodobacter spaeroides |

Enzymes exhibiting a 4-hydroxybutyrate activity (EC 1.1.1.61) can also convert malonic semialdehyde to 3-HP, as the chemical transformation is very similar. Table 8 below summarizes the relevant 4-hydroxybutyrate dehydrogenase gene information.

TABLE 8

| Gene | GenBank ID | GI | Organism |
|---|---|---|---|
| 4hbd | YP_726053.1 | 113867564 | Ralstonia eutropha H16 |
| 4hbd | AAA92347.1 | 347072 | Clostridium kluyveri DSM |
| | | | 555 |

In some embodiments, the present invention provides a non-naturally occurring microbial organism having a 3-HP pathway that includes at least one exogenous nucleic acid encoding a 3-HP pathway enzyme expressed in a sufficient amount to produce 3-HP. The pathway proceeds by way of malonyl-CoA. One exemplary exogenous nucleic acid in the 3-HP pathway is a dehydrogenase capable of converting oxaloacetate and CoA to malonyl-CoA. In some embodiments, this exogenous nucleic acid is a heterologous nucleic acid. In some embodiments, the microbial organism is cultured in a substantially anaerobic culture medium.

The invention additionally provides a method of producing 3-HP that includes culturing a non-naturally occurring microorganism having a biosynthetic pathway for the production of 3-hydroxypropionic acid via a malonyl-CoA intermediate. In some embodiments, the culture is performed under anaerobic conditions. In other embodiments, the culture is performed under aerobic conditions. The CoA-dependent dehydrogenation of oxaloacetate can be effected by naturally occurring 2-ketoacid dehydrogenases. In other embodiments, the 2-ketoacid dehydrogenases can be altered for increased activity on oxaloacetate. Such alterations can be introduced through a variety of directed and/or adaptive evolution methods.

As shown in FIG. 2, in the second biosynthetic step, oxaloacetate is converted to malonyl-CoA by an oxaloacetate dehydrogenase. An enzyme or enzyme complex catalyzing this particular transformation is not naturally occurring, however, this reaction can be accomplished by an enzyme in the 2-keto-acid dehydrogenase family. Enzymes in this family that catalyze similar transformations include 1) branched-chain 2-keto-acid dehydrogenase (EC 1.2.1.25), 2) alpha-ketoglutarate dehydrogenase (EC 1.2.1.52 and EC 1.2.1.-), and 3) the pyruvate dehydrogenase multienzyme complex (PDHC) (EC 1.2.1.-). These enzymes are multi-enzyme complexes that catalyze a series of partial reactions which result in acylating oxidative decarboxylation of 2-keto-acids. Each of the 2-keto-acid dehydrogenase complexes are part of intermediary metabolism, and enzyme activity can be tightly regulated (Fries et al., 2003 Biochemistry 42:6996-7002 (2003).) The enzymes share a complex but common structure composed of multiple copies of three catalytic components: alpha-ketoacid decarboxylase (E1), dihydrolipoamide acyltransferase (E2) and dihydrolipoamide dehydrogenase (E3). The E3 component is shared among all 2-keto-acid dehydrogenase complexes in an organism, while the E1 and E2 components are encoded by different genes. The enzyme components are present in numerous copies in the complex and utilize multiple cofactors to catalyze a directed sequence of reactions via substrate channeling. The overall size of these dehydrogenase complexes is very large, with molecular masses between 4 and 10 million Da.

Activity of enzymes in the 2-keto-acid dehydrogenase family can be low or limited under anaerobic conditions in organisms such as E. coli. Increased production of NADH (or NADPH) results in a redox-imbalance, and NADH itself serves as an inhibitor to enzyme function. Recent engineering efforts have increased the anaerobic activity of the E. coli pyruvate dehydrogenase complex (Kim et al., Appl. Environ. Microbiol. 73:1766-1771 (2007); Kim et al., J. Bacteriol. 190:3851-3858 (2008); Zhou et al., Biotechnol. Lett. 30:335-342 (2008)). For example, the inhibitory effect of NADH can be overcome by engineering an H322Y mutation in the E3 component (Kim et al., J. Bacteriol. 190:3851-3858 (2008)). Structural studies of individual components have been reported and show how they work together in a complex. These studies indicate the catalytic mechanisms and architecture of enzymes in this family (Aevarsson et al., Nat. Struct. Biol. 6:785-792 (1999); Zhou et al., Proc. Natl. Acad. Sci. U.S.A. 98:14802-14807 (2001)). The substrate specificity of the dehydrogenase complexes varies in different organisms, but generally branched-chain keto-acid dehydrogenases have the broadest substrate range.

Alpha-ketoglutarate dehydrogenase (AKGD) converts alpha-ketoglutarate to succinyl-CoA (FIG. 3B) and is the primary site of control of metabolic flux through the TCA cycle (Hansford, R. G., Curr. Top. Bioenerg. 10:217-278 (1980)). Encoded by genes sucA, sucB and lpd in E. coli, AKGD gene expression is downregulated under anaerobic conditions and during growth on glucose (Park et al., Mol. Microbiol. 15:473-482 (1995)). Although the substrate range of AKGD is narrow, structural studies of the catalytic core of the E2 component have indicated specific residues responsible for substrate specificity (Knapp et al., J. Mol. Biol. 280:655-668 (1998)). The Bacillus subtilis AKGD, encoded by odhAB (E1 and E2) and pdhD (E3, shared domain), is regulated at the transcriptional level and is dependent on the carbon source and growth phase of the organism (Resnekov et al., Mol. Gen. Genet. 234:285-296 (1992)). In yeast, the LPD1 gene encoding the E3 component is regulated at the transcriptional level by glucose (Roy and Dawes, J. Gen. Microbiol. 133:925-933 (1987)). The E1 component, encoded by KGD1, is also regulated by glucose and activated by the products of HAP2 and HAP3 (Repetto and Tzagoloff, Mol. Cell Biol. 9:2695-2705 (1989)). The AKGD enzyme complex, inhibited by products NADH and succinyl-CoA, is well-studied in mammalian systems, as impaired function of has been linked to several neurological diseases (Tretter and dam-Vizi, Philos. Trans. R. Soc. Lond B Biol. Sci 360:2335-2345(2005)). The various genes encoding AKGD are shown below in Table 9.

TABLE 9

| | | | |
|---|---|---|---|
| sucA | NP_415254.1 | 16128701 | Escherichia coli str. K12 substr. |
| | | | MG1655 |
| sucB | NP_415255.1 | 16128702 | Escherichia coli str. K12 substr. |
| | | | MG1655 |
| lpd | NP_414658.1 | 16128109 | Escherichia coli str. K12 substr. |
| | | | MG1655 |
| odhA | P23129.2 | 51704265 | Bacillus subtilis |
| odhB | P16263.1 | 129041 | Bacillus subtilis |
| pdhD | P21880.1 | 118672 | Bacillus subtilis |
| KGD1 | NP_012141.1 | 6322066 | Saccharomyces cerevisiae |
| KGD2 | NP_010432.1 | 6320352 | Saccharomyces cerevisiae |
| LPD1 | NP_116635.1 | 14318501 | Saccharomyces cerevisiae |

Branched-chain 2-keto-acid dehydrogenase complex (BCKAD), also known as 2-oxoisovalerate dehydrogenase, (EC 1.2.1.25) participates in branched-chain amino acid degradation pathways, converting 2-keto acids derivatives of valine, leucine and isoleucine to their acyl-CoA derivatives and CO₂. The complex has been studied in many organisms including Bacillus subtilis (Wang et al., Eur. J. Biochem 213:1091-1099 (1993)), Rattus norvegicus (Namba et al., J. Biol. Chem 244:4437-4447 (1969)) and Pseudomonas putida (Sokatch et al., J. Bacteriol. 148:647-652 (1981)). In Bacillus subtilis the enzyme is encoded by genes pdhD (E3 component), bfmBB (E2 component), bfmBAA and bfmBAB (E1 component) (Wang et al., Eur. J. Biochem 213:1091-1099 (1993)). In mammals, the complex is regulated by phosphorylation by specific phosphatases and protein kinases. The complex has been studied in rat hepatocites (Chicco et al., J. Biol. Chem. 269:19427-19434 (1994)) and is encoded by genes Bckdha (E1 alpha), Bckdhb (E1 beta), Dbt (E2), and Dld (E3). The E1 and E3 components of the Pseudomonas putida BCKAD complex have been crystallized (Aevarsson et al., Nat. Struct. Biol. 6:785-792 (1999); Mattevi et al., Science 255:1544-1550 (1992)) and the enzyme complex has been studied (Sokatch et al., J. Bacteriol. 148:647-652 (1981)). Transcription of the P. putida BCKAD genes is activated by the gene product of bkdR (Hester et al., Eur. J. Biochem 233:828-836 (1995)). In some organisms including Rattus norvegicus (Paxton et al., Biochem. J. 234:295-303 (1986)) and Saccharomyces cerevisiae (Sinclair et al., Biochem. Mol. Biol. Int. 31:911-922 (1993)), this complex has been shown to have a broad substrate range that includes linear oxo-acids such as 2-oxobutanoate (FIG. 3C) and alpha-ketoglutarate (FIG. 3B), in addition to the branched-chain amino acid precursors. The active site of the bovine BCKAD was engineered to favor alternate substrate acetyl-CoA (Meng and Chuang, Biochemistry 33:12879-12885 (1994)). The various genes encoding BCKADs are summarized below in Table 10.

TABLE 10

| | | | |
|---|---|---|---|
| bfmBB | NP_390283.1 | 16079459 | Bacillus subtilis |
| bfmBAA | NP_390285.1 | 16079461 | Bacillus subtilis |
| bfmBAB | NP_390284.1 | 16079460 | Bacillus subtilis |
| pdhD | P21880.1 | 118672 | Bacillus subtilis |
| lpdV | P09063.1 | 118677 | Pseudomonas putida |
| bkdB | P09062.1 | 129044 | Pseudomonas putida |
| bkdA1 | NP_746515.1 | 26991090 | Pseudomonas putida |
| bkdA2 | NP_746516.1 | 26991091 | Pseudomonas putida |
| Bckdha | NP_036914.1 | 77736548 | Rattus norvegicus |
| Bckdhb | NP_062140.1 | 158749538 | Rattus norvegicus |
| Dbt | NP_445764.1 | 158749632 | Rattus norvegicus |
| Dld | NP_955417.1 | 40786469 | Rattus norvegicus |

The pyruvate dehydrogenase complex (EC 1.2.1.-), catalyzing the conversion of pyruvate to acetyl-CoA, has been studied. In the E. coli enzyme, specific residues in the E1 component are responsible for substrate specificity (Bisswanger, H., J Biol Chem 256:815-822 (1981); Bremer, J.,. Eur. J Biochem 8:535-540 (1969); Gong et al., J Biol Chem 275:13645-13653 (2000)). As mentioned previously, enzyme engineering efforts have improved the E. coli PDH enzyme activity under anaerobic conditions (Kim et al., Appl. Environ. Microbiol. 73:1766-1771 (2007); Kim et al., J. Bacteriol. 190:3851-3858 (2008); Zhou et al., Biotechnol. Lett. 30:335-342 (2008)). In contrast to the E. coli PDH, the B. subtilis complex is active and required for growth under anaerobic conditions (Nakano et al., J. Bacteriol. 179:6749-6755 (1997)). The Klebsiella pneumoniae PDH, characterized during growth on glycerol, is also active under anaerobic conditions (Menzel et al., J. Biotechnol. 56:135-142 (1997)). Crystal structures of the enzyme complex from bovine kidney (Zhou et al., Proc. Natl. Acad. Sci. U.S.A 98:14802-14807 (2001) and the E2 catalytic domain from Azotobacter vinelandii are available (Mattevi et al., Science 255:1544-1550 (1992)). Some mammalian PDH enzymes complexes can react on alternate substrates such as 2-oxobutanoate (FIG. 3C), although comparative kinetics of Rattus norvegicus PDH and BCKAD indicate that BCKAD has higher activity on 2-oxobutanoate as a substrate (Paxton et al., Biochem. J. 234:295-303 (1986)). The gene information for the various pyruvate dehydrogenase complexes is summarized below in Table 11.

TABLE 11

| | | | |
|---|---|---|---|
| aceE | NP_414656.1 | 16128107 | Escherichia coli str. K12 |
| | | | substr. MG1655 |
| aceF | NP_414657.1 | 16128108 | Escherichia coli str. K12 |
| | | | substr. MG1655 |
| lpd | NP_414658.1 | 16128109 | Escherichia coli str. K12 |
| | | | substr. MG1655 |
| pdhA | P21881.1 | 3123238 | Bacillus subtilis |
| pdhB | P21882.1 | 129068 | Bacillus subtilis |
| pdhC | P21883.2 | 129054 | Bacillus subtilis |
| pdhD | P21880.1 | 118672 | Bacillus subtilis |
| aceE | YP_001333808.1 | 152968699 | Klebsiella pneumonia |
| | | | MGH78578 |
| aceF | YP_001333809.1 | 152968700 | Klebsiella pneumonia |
| | | | MGH78578 |
| lpdA | YP_001333810.1 | 152968701 | Klebsiella pneumonia |
| | | | MGH78578 |
| Pdha1 | NP_001004072.2 | 124430510 | Rattus norvegicus |
| Pdha2 | NP_446446.1 | 16758900 | Rattus norvegicus |
| Dlat | NP_112287.1 | 78365255 | Rattus norvegicus |
| Dld | NP_955417.1 | 40786469 | Rattus norvegicus |

An alternative to the multienzyme 2-keto-acid dehydrogenase complexes described above, some anaerobic organisms utilize enzymes in the 2-ketoacid oxidoreductase family (OFOR) to catalyze acylating oxidative decarboxylation of 2-keto-acids. Unlike the dehydrogenase complexes, these enzymes contain iron-sulfur clusters, utilize different cofactors, and use ferredoxin or flavodoxin as electron acceptors in lieu of NAD(P)H. While most enzymes in this family are specific to pyruvate as a substrate (POR) some 2-keto-acid:ferredoxin oxidoreductases have been shown to accept a broad range of 2-ketoacids as substrates including alpha-ketoglutarate and 2-oxobutanoate (Fukuda and Wakagi, Biochim. Biophys. Acta 1597:74-80 (2002); Zhang et al., J. Biochem. 120:587-599 (1996)). One such enzyme is the OFOR from the thermoacidophilic archaeon Sulfolobus tokodaii 7, which contains an alpha and beta subunit encoded by gene ST2300 (Fukuda and Wakagi, Biochim. Biophys. Acta 1597:74-80 (2002); Zhang et al., J. Biochem 120:587-599 (1996)). A plasmid-based expression system has been developed for efficiently expressing this protein in E. coli (Fukuda et al., Eur. J. Biochem 268:5639-5646 (2001) and residues involved in substrate specificity were determined (Fukuda and Wakagi, Biochim. Biophys. Acta 1597:74-80 (2002)). Two OFORs from Aeropyrum pernix str. K1 have also been recently cloned into E. coli, characterized, and found to react with a broad range of 2-oxoacids (Nishizawa et al., FEBS Lett. 579:2319-2322 (2005)). The gene sequences of these OFOR candidates are available, although they do not have GenBank identifiers assigned to date. There is bioinformatic evidence that similar enzymes are present in all archaea, some anaerobic bacteria and amitochondrial eukarya (Fukuda et al., Biochim. Biophys. Acta 1597:74-80 (2002)). From an energetic standpoint, reduced ferredoxin can be used to generate NADH by ferredoxin-NAD reductase (Petitdemange et al., Biochim. Biophys. Acta 421:334-337 (1976)). Also, since most of the enzymes are designed to operate under anaerobic conditions, less enzyme engineering is required relative to enzymes in the 2-keto-acid dehydrogenase complex family. The OFOR gene from Sulfolobus tokodaii 7 is designated ST2300 (NP_{—}378302.1).

Yet another alternative for converting oxaloacetate to malonyl-CoA is an enzyme with oxaloacetate formate-lyase activity. Enzymes in this class acylate ketoacids to their corresponding CoA derivatives with concomitant release of formate. Two exemplary formate-lyase enzymes for catalyzing this conversion are pyruvate formate-lyase and ketoacid formate-lyase. Pyruvate formate-lyase (PFL, EC 2.3.1.54), encoded by pflB in E. coli, naturally converts pyruvate into acetyl-CoA and formate. The active site of PFL contains a catalytically essential glycyl radical that is posttranslationally activated under anaerobic conditions by PFL-activating enzyme (PFL-AE, EC 1.97.1.4) encoded by pflA (Knappe et al., Proc. Natl. Acad. Sci U.S.A. 81:1332-1335 (1984); and Wong et al., Biochemistry 32:14102-14110 (1993)). A pyruvate formate-lyase from Archaeglubus fulgidus encoded by pflD has been cloned, expressed in E. coli and characterized (Lehtio, L. and A. Goldman, Protein Eng Des Sel 17:545-552 (2004)). The crystal structures of the A. fulgidus and E. coli enzymes have been resolved (Lehtio et al., J Mol. Biol. 357:221-235 (2006)). Additional PFL and PFL-AE candidates are found in Clostridium pasteurianum (Weidner, G. and G. Sawers, J. Bacteriol. 178:2440-2444 (1996)) and the eukaryotic alga Chlamydomonas reinhardtii (Cary et al., Appl. Environ. Microbiol 56:1576-1583 (1990)). Keto-acid formate-lyase (EC 2.3.1.-), also known as 2-ketobutyrate formate-lyase (KFL) and pyruvate formate-lyase 4, is the gene product of tdcE in E. coli. This enzyme catalyzes the conversion of 2-ketobutyrate to propionyl-CoA and formate during anaerobic threonine degradation, and can also substitute for pyruvate formate-lyase in anaerobic catabolism (Simanshu et al., J Biosci. 32:1195-1206 (2007)). The enzyme is oxygen-sensitive and, like PflB, requires post-translational modification by PFL-AE to activate a glycyl radical in the active site (Hesslinger et al., Mol. Microbiol. 27:477-492 (1998)). The gene information for the various enzymes described above is summarized below in Table 12.

TABLE 12

| | | | |
|---|---|---|---|
| pflB | 16128870 | NP_415423.1 | Escherichia coli |
| pflA | 16128869 | NP_415422.1 | Escherichia coli |
| tdcE | 48994926 | AAT48170.1 | Escherichia coli |
| pflD | 11499044 | NP_070278.1 | Archaeglubus fulgidus |
| pfl | 2500058 | Q46266.1 | Clostridium pasteurianum |
| act | 1072362 | CAA63749.1 | Clostridium pasteurianum |
| pfl1 | 159462978 | XP_001689719.1 | Chlamydomonas reinhardtii |
| pflA1 | 159485246 | XP_001700657.1 | Chlamydomonas reinhardtii |

In some embodiments, the non-naturally occurring microbial organism possessing the malonyl-CoA to 3-HP pathway includes an exogenous nucleic acid encoding a phosphoenolpyruvate carboxykinase. The first step shown in FIG. 2 is the conversion of phosphoenolpyruvate to oxaloacetate by phosphoenolpyruvate carboxykinase (PPCK). Note that PPCK results in the production of one ATP molecule per conversion of PEP to oxaloacetate. Thus PEP carboxykinase is beneficial for the production of 3-hydroxypropionic acid in a high yield under anaerobic conditions because provides favorable energetics to the engineered organism through the production of 3-hydroxypropionic acid. Any of the exemplary PPCK enzymes discussed above with reference to FIG. 1 can be used in the pathway can be used to generate oxaloacetate.

In still further embodiments, the non-naturally occurring microbial organism includes an exogenous nucleic acid encoding a bifunctional malonyl-CoA reductase with aldehyde dehydrogenase and alcohol dehydrogenase functionality. Specifically, the reduction of malonyl-CoA to 3-HP can be accomplished by a bifunctional malonyl-CoA reductase with aldehyde dehydrogenase and alcohol dehydrogenase functionality. An NADPH-dependent enzyme with this activity has been characterized in Chloroflexus aurantiacus where it participates in the 3-hydroxypropionate cycle (Hugler et al., J. Bacteriol. 184:2404-2410 (2002); Strauss and Fuchs, Eur. J. Biochem 215:633-643 (1993)). This enzyme, with a mass of 300 kDa, is highly substrate-specific and shows little sequence similarity to other known oxidoreductases. (Hugler et al., J. Bacteriol. 184:2404-2410 (2002); Strauss and Fuchs, Eur. J. Biochem 215:633-643 (1993)). There is bioinformatic evidence that other organisms may have similar pathways (Klatt et al., Environ. Microbiol. 9:2067-2078 (2007)). Enzymes in other organisms including Roseiflexus castenholzii, Erythrobacter sp. NAP1 and marine gamma proteobacterium HTCC2080 can be inferred by sequence similarity. The various genes are summarized in Table 13 below.

TABLE 13

| | | | |
|---|---|---|---|
| mcr | AAS20429.1 | 42561982 | Chloroflexus |
| | | | aurantiacus |
| Rcas_2929 | YP_001433009.1 | 156742880 | Roseiflexus |
| | | | castenholzii |
| NAP1_02720 | ZP_01039179.1 | 85708113 | Erythrobacter |
| | | | sp. NAP1 |
| MGP2080_00535 | ZP_01626393.1 | 119504313 | marine gamma |
| | | | proteobacterium |
| | | | HTCC2080 |

Alternatively, the reduction of malonyl-CoA to 3-HP can be catalyzed by two separate enzymes: a CoA-acylating malonate semialdehyde dehydrogenase and a 3-hydroxypropionate dehydrogenase). By this route, malonyl-CoA is first reduced to malonate semialdehyde (MSA) by malonate-semialdehyde dehydrogenase or malonyl-CoA reductase. MSA is subsequently converted to 3-HP by 3-HP-dehydrogenase. Malonyl-CoA reductase is an enzyme used in autotrophic carbon fixation via the 3-hydroxypropionate cycle in thermoacidophilic archael bacteria (Berg et al., Science 318:1782-1786 (2007); Thauer, R. K., Science 318:1732-1733 (2007). The enzyme which utilizes NADPH as a cofactor, has been characterized in Metallosphaera and Sulfolobus spp (Alber et al., J. Bacteriol. 188:8551-8559; (2006); Hugler et al., J. Bacteriol. 184:2404-2410 (2002)). The enzyme encoded by Msed_{—}0709 in Metallosphaera sedula is known to convert malonyl-CoA to malonic semialdehdye and operate in the direction of interest (Alber et al., J. Bacteriol. 188:8551-8559 (2006); Berg et al., Science 318:1782-1786 (2007)). A gene encoding a malonyl-CoA reductase from Sulfolobus tokodaii was cloned and heterologously expressed in E. coli (Alber et al., J. Bacteriol. 188:8551-8559 (2006)). Although the aldehyde dehydrogenase functionality of these enzymes is similar to the bifunctional dehydrogenase from Chloroflexus aurantiacus, there is little sequence similarity. Both malonyl-CoA reductase enzyme candidates have high sequence similarity to aspartate-semialdehyde dehydrogenase, an enzyme catalyzing the reduction and concurrent dephosphorylation of aspartyl-4-phosphate to aspartate semialdehyde. Additional gene candidates are present and identifiable by sequence homology to proteins in other organisms including Sulfolobus solfataricus and Sulfolobus acidocaldarius. The various genes are summarize below in Table 14.

TABLE 14

| | | | |
|---|---|---|---|
| Msed_0709 | YP_001190808.1 | 146303492 | Metallosphaera sedula |
| mcr | NP_378167.1 | 15922498 | Sulfolobus tokodaii |
| asd-2 | NP_343563.1 | 15898958 | Sulfolobus solfataricus |
| Saci_2370 | YP_256941.1 | 70608071 | Sulfolobus |
| | | | acidocaldarius |

The subsequent conversion of malonic semialdehyde to 3-HP can be accomplished by an enzyme with 3-HP dehydrogenase activity as described herein above with reference to FIG. 1.

In some embodiments, the present invention provides a non-naturally occurring microbial organism having a 3-HP pathway that includes at least one exogenous nucleic acid encoding a 3-HP pathway enzyme expressed in a sufficient amount to produce 3-HP. The pathway proceeds by way of malate. One exemplary exogenous nucleic acid in the malate pathway is a decarboxylase capable of converting malate to 3-HP. In some embodiments, this exogenous nucleic acid is a heterologous nucleic acid. In some embodiments, the microbial organism is cultured in a substantially anaerobic culture medium.

The invention additionally provides a method of producing 3-HP that includes culturing a non-naturally occurring microorganism having a biosynthetic pathway for the production of 3-hydroxypropionic acid via a malate intermediate. In some embodiments, the culture is performed under anaerobic conditions. In other embodiments, the culture is performed under aerobic conditions. The decarboxylation of malate can be effected by naturally occurring enzymes with malate decarboxylase activity. In other embodiments, the decarboxylases can be altered for increased activity on malate. Such alterations can be introduced through a variety of directed and/or adaptive evolution methods.

As shown in FIG. 4, 3-HP is produced from malate derived from phosphoenolpyruvate. Phosphoenolpyruvate is carboxylated to form oxaloacetate as described above. Oxaloacetate is then converted to malate by malate dehydrogenase, an enzyme that participates in the TCA cycles of many organisms. In the third biosynthetic step, malate is converted to 3-HP by malate decarboxylase. An enzyme with this functionality is not known to occur naturally. A potential enzyme candidate for a 3-HP-forming malate decarboxylase is acetolactate decarboxylase (EC 4.1.1.5). This enzyme participates in citrate catabolism and branched-chain amino acid biosynthesis, converting 2-acetolactate to acetoin. In Lactococcus lactis the enzyme is composed of six subunits, encoded by gene aldB, and is activated by valine, leucine and isoleucine (Goupil et al., Appl. Environ. Microbiol. 62:2636-2640 (1996); Goupil-Feuillerat et al. J. Bacteriol. 182:5399-5408 (2000)). This enzyme has been overexpressed and characterized in E. coli (Phalip et al., FEBS Lett. 351:95-99 (1994)). In other organisms the enzyme is a dimer, encoded by aldC in Streptococcus thermophilus (Monnet et al. Lett. Appl. Microbiol. 36:399-405 (2003)), aldB in Bacillus brevis ((Diderichsen et al. J. Bacteriol. 172:4315-4321 (1990); Najmudin et al., Acta Chrystallogr. D. Biol. Crystallogr. 59:1073-1075 (2003)) and budA from Enterobacter aerogenes (Diderichsen et al. J. Bacteriol. 172:4315-4321 (1990)). The enzyme from Bacillus brevis was cloned and overexpressed in Bacillus subtilis and characterized crystallographically (Najmudin et al., Acta Chrystallogr. D. Biol. Crystallogr. 59:1073-1075 (2003)). Additionally, the enzyme from Leuconostoc lactis has been purified and characterized but the gene has not been isolated (O'Sullivan et al. FEMS Micriobiol. Lett. 194:245-249 (2001)).

TABLE 15

| | | | |
|---|---|---|---|
| aldB | NP_267384.1 | 15673210 | Lactococcus lactis |
| aldC | Q8L208 | 75401480 | Streptococcus thermophilus |
| aldB | P23616.1 | 113592 | Bacillus brevis |
| budA | P05361.1 | 113593 | Enterobacter aerogenes |

The invention is described herein with general reference to the metabolic reaction, reactant or product thereof, or with specific reference to one or more nucleic acids or genes encoding an enzyme associated with or catalyzing, or a protein associated with, the referenced metabolic reaction, reactant or product. Unless otherwise expressly stated herein, those skilled in the art will understand that reference to a reaction also constitutes reference to the reactants and products of the reaction. Similarly, unless otherwise expressly stated herein, reference to a reactant or product also references the reaction, and reference to any of these metabolic constituents also references the gene or genes encoding the enzymes that catalyze or proteins involved in the referenced reaction, reactant or product. Likewise, given the well known fields of metabolic biochemistry, enzymology and genomics, reference herein to a gene or encoding nucleic acid also constitutes a reference to the corresponding encoded enzyme and the reaction it catalyzes or a protein associated with the reaction as well as the reactants and products of the reaction.

The non-naturally occurring microbial organisms of the invention can be produced by introducing expressible nucleic acids encoding one or more of the enzymes or proteins participating in one or more 3-HP biosynthetic pathways. Depending on the host microbial organism chosen for biosynthesis, nucleic acids for some or all of a particular 3-HP biosynthetic pathway can be expressed. For example, if a chosen host is deficient in one or more enzymes or proteins for a desired biosynthetic pathway, then expressible nucleic acids for the deficient enzyme(s) or protein(s) are introduced into the host for subsequent exogenous expression. Alternatively, if the chosen host exhibits endogenous expression of some pathway genes, but is deficient in others, then an encoding nucleic acid is needed for the deficient enzyme(s) or protein(s) to achieve 3-HP biosynthesis. Thus, a non-naturally occurring microbial organism of the invention can be produced by introducing exogenous enzyme or protein activities to obtain a desired biosynthetic pathway or a desired biosynthetic pathway can be obtained by introducing one or more exogenous enzyme or protein activities that, together with one or more endogenous enzymes or proteins, produces a desired product such as 3-HP.

Depending on the 3-HP biosynthetic pathway, constituents of a selected host microbial organism, the non-naturally occurring microbial organisms of the invention will include at least one exogenously expressed 3-HP pathway-encoding nucleic acid and up to all encoding nucleic acids for one or more 3-HP biosynthetic pathways. For example, 3-HP biosynthesis can be established in a host deficient in a pathway enzyme or protein through exogenous expression of the corresponding encoding nucleic acid. In a host deficient in all enzymes or proteins of a 3-HP pathway, exogenous expression of all enzyme or proteins in the pathway can be included, although it is understood that all enzymes or proteins of a pathway can be expressed even if the host contains at least one of the pathway enzymes or proteins. For example, exogenous expression of all enzymes or proteins in a pathway for production of 3-HP can be included, such as a decarboxylase.

Given the teachings and guidance provided herein, those skilled in the art will understand that the number of encoding nucleic acids to introduce in an expressible form will, at least, parallel the 3-HP pathway deficiencies of the selected host microbial organism. Therefore, a non-naturally occurring microbial organism of the invention can have one, two, three, up to all nucleic acids encoding the enzymes or proteins constituting a 3-HP biosynthetic pathway disclosed herein. In some embodiments, the non-naturally occurring microbial organisms also can include other genetic modifications that facilitate or optimize 3-HP biosynthesis or that confer other useful functions onto the host microbial organism. One such other functionality can include, for example, augmentation of the synthesis of one or more of the 3-HP pathway precursors such as oxaloacetate and/or malonate semialdehyde.

Generally, a host microbial organism is selected such that it produces the precursor of a 3-HP pathway, either as a naturally produced molecule or as an engineered product that either provides de novo production of a desired precursor or increased production of a precursor naturally produced by the host microbial organism. For example, oxaloacetate or malonate semialdehyde is produced naturally in a host organism such as E. coli. A host organism can be engineered to increase production of a precursor, as disclosed herein. In addition, a microbial organism that has been engineered to produce a desired precursor can be used as a host organism and further engineered to express enzymes or proteins of a 3-HP pathway.

In some embodiments, a non-naturally occurring microbial organism of the invention is generated from a host that contains the enzymatic capability to synthesize 3-HP. In this specific embodiment it can be useful to increase the synthesis or accumulation of a 3-HP pathway product to, for example, drive 3-HP pathway reactions toward 3-HP production. Increased synthesis or accumulation can be accomplished by, for example, overexpression of nucleic acids encoding one or more of the above-described 3-HP pathway enzymes or proteins. Overexpression of the enzyme or enzymes and/or protein or proteins of the 3-HP pathway can occur, for example, through exogenous expression of the endogenous gene or genes, or through exogenous expression of the heterologous gene or genes. Therefore, naturally occurring organisms can be readily generated to be non-naturally occurring microbial organisms of the invention, for example, producing 3-HP, through overexpression of one, two, three, that is, up to all nucleic acids encoding 3-HP biosynthetic pathway enzymes or proteins. In addition, a non-naturally occurring organism can be generated by mutagenesis of an endogenous gene that results in an increase in activity of an enzyme in the 3-HP biosynthetic pathway.

In particularly useful embodiments, exogenous expression of the encoding nucleic acids is employed. Exogenous expression confers the ability to custom tailor the expression and/or regulatory elements to the host and application to achieve a desired expression level that is controlled by the user. However, endogenous expression also can be utilized in other embodiments such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring microbial organism.

It is understood that, in methods of the invention, any of the one or more exogenous nucleic acids can be introduced into a microbial organism to produce a non-naturally occurring microbial organism of the invention. The nucleic acids can be introduced so as to confer, for example, a 3-HP biosynthetic pathway onto the microbial organism. Alternatively, encoding nucleic acids can be introduced to produce an intermediate microbial organism having the biosynthetic capability to catalyze some of the required reactions to confer 3-HP biosynthetic capability. For example, a non-naturally occurring microbial organism having 3-HP biosynthetic pathway can comprise at least one exogenous nucleic acid encoding desired enzymes or proteins, such as a decarboxylase, and the like.

In addition to the biosynthesis of 3-HP as described herein, the non-naturally occurring microbial organisms and methods of the invention also can be utilized in various combinations with each other and with other microbial organisms and methods well known in the art to achieve product biosynthesis by other routes. For example, one alternative to produce 3-HP other than use of the 3-HP producers is through addition of another microbial organism capable of converting a 3-HP pathway intermediate to 3-HP. One such procedure includes, for example, the fermentation of a microbial organism that produces a 3-HP pathway intermediate. The 3-HP pathway intermediate can then be used as a substrate for a second microbial organism that converts the 3-HP pathway intermediate to 3-HP. The 3-HP pathway intermediate can be added directly to another culture of the second organism or the original culture of the 3-HP pathway intermediate producers can be depleted of these microbial organisms by, for example, cell separation, and then subsequent addition of the second organism to the fermentation broth can be utilized to produce the final product without intermediate purification steps.

In other embodiments, the non-naturally occurring microbial organisms and methods of the invention can be assembled in a wide variety of subpathways to achieve biosynthesis of, for example, 3-HP. In these embodiments, biosynthetic pathways for a desired product of the invention can be segregated into different microbial organisms, and the different microbial organisms can be co-cultured to produce the final product. In such a biosynthetic scheme, the product of one microbial organism is the substrate for a second microbial organism, and so on, until the final product is synthesized. For example, the biosynthesis of 3-HP can be accomplished by constructing a microbial organism that contains biosynthetic pathways for conversion of one pathway intermediate to another pathway intermediate or the product. Alternatively, 3-HP also can be bio synthetically produced from microbial organisms through co-culture or co-fermentation using two organisms in the same vessel, where the first microbial organism produces a malonate semialdehyde intermediate and the second microbial organism converts the intermediate to 3-HP.

Microorganisms capable of directly producing 3-HP are constructed by introducing genes encoding decarboxylase enzymes into the strains engineered for maximal oxaloacetate production. The following example describes the creation of a microbial organism that can produce 3-HP from renewable feedstocks such as glucose or sucrose.

To generate an E. coli strain engineered to produce 3-HP, nucleic acids encoding the decarboxylase enzymes are cloned and expressed in E. coli capable of overproducing oxaloacetate using well known molecular biology techniques and recombinant and detection methods well known in the art. Such methods are described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999).

A 3-HP producing strain is constructed, by cloning the individual PPCK, decarboxylase and malonate dehydrogenase genes into pZA33 or pZE13 vectors (Expressys, Ruelzheim, Germany) under the IPTG-titratable PA1/lacO promoter. The plasmids are transformed into the oxaloacetate overproducing E. coli strain using standard methods such as electroporation. The resulting genetically engineered organism is cultured in glucose-containing medium following procedures well known in the art (see, for example, Sambrook et al., supra, 2001). Expression of the decarboxylase genes are corroborated using methods well known in the art for determining polypeptide expression or enzymatic activity, including for example, Northern blots, PCR amplification of mRNA, immunoblotting, and the like. Enzymatic activities of the expressed enzymes are confirmed using assays specific for the individual activities. The ability of the engineered E. coli strain to produce 3-HP is confirmed using HPLC, gas chromatography-mass spectrometry (GCMS) and/or liquid chromatography-mass spectrometry (LCMS).

Microbial strains engineered to have a functional 3-HP synthesis pathway are further augmented by optimization for efficient utilization of the pathway. Briefly, the engineered strain is assessed to determine whether exogenous genes are expressed at a rate limiting level. Flux analysis using ¹³C-labeled glucose is performed to assess bottlenecks in the system. Expression is increased for enzymes produced at low levels and that limit the flux through the pathway by, for example, introduction of additional gene copy numbers or changes to the promoter and ribosome binding sites.

To generate better 3-HP producers, metabolic modeling is utilized to optimize growth conditions. Modeling is also used to design gene knockouts that additionally optimize utilization of the pathway, as described above. Modeling analysis allows reliable predictions of the effects on cell growth of shifting the metabolism towards more efficient production of 3-HP. Adaptive evolution is performed to improve both growth and production characteristics (Fong and Palsson, Nat Genet. 36:1056-1058 (2004)). Based on the results, subsequent rounds of modeling, genetic engineering and adaptive evolution can be applied to the 3-HP producer to further increase production.

For large-scale production of 3-HP, the above organism is cultured in a fermenter using a medium known in the art to support growth of the organism under anaerobic conditions. Fermentations are performed in either a batch, fed-batch or continuous manner. Anaerobic conditions are maintained by first sparging the medium with nitrogen and then sealing the culture vessel, for example, flasks can be sealed with a septum and crimp-cap. Microaerobic conditions also can be utilized by providing a small hole in the septum for limited aeration. The pH of the medium is maintained in the optimum range by addition of acids such as H₂SO₄ or bases such as NaOH or Na₂CO₃. The growth rate is determined by measuring optical density using a spectrophotometer (600 nm) and the glucose uptake rate by monitoring carbon source depletion over time. Byproducts such as undesirable alcohols, organic acids, and residual glucose can be quantified by HPLC (Shimadzu, Columbia Md.), for example, using an Aminex® series of HPLC columns (for example, HPX-87 series) (BioRad, Hercules Calif.), using a refractive index detector for glucose and alcohols, and a UV detector for organic acids (Lin et al., Biotechnol. Bioeng. 775-779 (2005)).

E. coli and other microorganisms are known to possess fatty acid and organic acid degradation pathways that could lead to 3-HP degradation. While fermentative production of 3-HP under anaerobic conditions should not be accompanied by degradation, should product degradation be observed, the pathways responsible for product degradation will be deleted.

Given the teachings and guidance provided herein, those skilled in the art will understand that a wide variety of combinations and permutations exist for the non-naturally occurring microbial organisms and methods of the invention together with other microbial organisms, with the co-culture of other non-naturally occurring microbial organisms having subpathways and with combinations of other chemical and/or biochemical procedures well known in the art to produce 3-HP.

Sources of encoding nucleic acids for a 3-HP pathway enzyme or protein can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. Exemplary species for such sources include, for example, Escherichia coli, Candida albicans, Candida boidinii, Aspergillus terreus, Pseudomonas sp. CF600, Pseudomonas putida, Ralstonia eutropha JMP134, Saccharomyces cerevisae, Lactobacillus plantarum, Klebsiella oxytoca, Bacillus subtilis, Bacillus pumilus, Pedicoccus pentosaceus, as well as other exemplary species disclosed herein or available as source organisms for corresponding genes. However, with the complete genome sequence available for now more than 550 species (with more than half of these available on public databases such as the NCBI), including 395 microorganism genomes and a variety of yeast, fungi, plant, and mammalian genomes, the identification of genes encoding the requisite 3-HP biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations enabling biosynthesis of 3-HP described herein with reference to a particular organism such as E. coli can be readily applied to other microorganisms, including prokaryotic and eukaryotic organisms alike. Given the teachings and guidance provided herein, those skilled in the art will know that a metabolic alteration exemplified in one organism can be applied equally to other organisms.

In some instances, such as when an alternative 3-HP biosynthetic pathway exists in an unrelated species, 3-HP biosynthesis can be conferred onto the host species by, for example, exogenous expression of a paralog or paralogs from the unrelated species that catalyzes a similar, yet non-identical metabolic reaction to replace the referenced reaction. Because certain differences among metabolic networks exist between different organisms, those skilled in the art will understand that the actual gene usage between different organisms can differ. However, given the teachings and guidance provided herein, those skilled in the art also will understand that the teachings and methods of the invention can be applied to all microbial organisms using the cognate metabolic alterations to those exemplified herein to construct a microbial organism in a species of interest that will synthesize 3-HP.

Host microbial organisms can be selected from, and the non-naturally occurring microbial organisms generated in, for example, bacteria, yeast, fungus or any of a variety of other microorganisms applicable to fermentation processes. Exemplary bacteria include species selected from Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Exemplary yeasts or fungi include species selected from Saccharomyces cerevisiae, Schizosaccharomyces pombe, Kluyveromyces lactis, Kluyveromyces marxianus, Aspergillus terreus, Aspergillus niger and Pichia pastoris. E. coli is a particularly useful host organism since it is a well characterized microbial organism suitable for genetic engineering. Other particularly useful host organisms include yeast such as Saccharomyces cerevisiae.

Methods for constructing and testing the expression levels of a non-naturally occurring 3-HP-producing host can be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999).

Exogenous nucleic acid sequences involved in a pathway for production of 3-HP can be introduced stably or transiently into a host cell using techniques well known in the art including, but not limited to, conjugation, electroporation, chemical transformation, transduction, transfection, and ultrasound transformation. For exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

An expression vector or vectors can be constructed to include one or more 3-HP biosynthetic pathway encoding nucleic acids as exemplified herein operably linked to expression control sequences functional in the host organism. Expression vectors applicable for use in the microbial host organisms of the invention include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

In some embodiments, a method for producing 3-HP includes culturing a non-naturally occurring microbial organism having a 3-HP pathway. The pathway includes at least one exogenous nucleic acid encoding a 3-HP pathway enzyme expressed in a sufficient amount to produce 3-HP under conditions and for a sufficient period of time to produce 3-HP. Ideally, the non-naturally occurring microbial organism is in a substantially anaerobic culture medium as described above.

The 3-HP pathway includes an oxaloacetate decarboxylase gene introduced into an organism that is engineered to produce high levels of oxaloacetate under anaerobic conditions from carbon substrates such as glucose, sucrose, CO, CO₂, and methanol as well as other sources disclosed herein. Expression of active decarboxylases for the production of chemicals previously has been demonstrated in E. coli (Sariaslani, F. S., Annu. Rev. Microbiol. 61:51-69 (2007)). In this scenario, decarboxylation of oxaloacetate occurs intracellularly and following reduction of the resultant malonate semialdehyde product, 3-HP is produced and is secreted from the cell and recovered through standard methods employed for acid separation and purification.

An important consideration for bioprocessing is whether to use a batch or continuous fermentation scheme. One difference between the two schemes that will influence the amount of product produced is the presence of a preparation, lag, and stationary phase for the batch scheme in addition to the exponential growth phase. In contrast, continuous processes are kept in a state of constant exponential growth and, if properly operated, can run for many months at a time. For growth-associated and mixed-growth-associated product formation, continuous processes provide much higher productivities (i.e., dilution rate times cell mass) due to the elimination of the preparation, lag, and stationary phases. For example, given the following reasonable assumptions:

Monod kinetics (i.e., μ=μₘ·S/(Kₛ+S))

μₘ=1.0 hr⁻¹

final cell concentration/initial cell concentration=20

tₚᵣₑₚ+t_{lag}+tₛₜₐₜ=5 hr

feed concentration of limiting nutrient>>Ks

increased productivity from a continuous process has been estimated at 8-fold, Shuler et al, Prentice Hall, Inc.: Upper Saddle River, N.J., 245-247.

Despite the overwhelming advantage in productivity, many more batch processes are in operation than continuous processes for a number of reasons. First, for non-growth associated product formation (e.g., penicillin), the productivity of a batch system may significantly exceed that of a continuous process because the latter would have to operate at very low dilution rates. Next, production strains generally have undergone modifications to their genetic material to improve their biochemical or protein production capabilities. These specialized strains are likely to grow less rapidly than their parental complements whereas continuous processes such those employing chemostats (fermenters operated in continuous mode) impose large selection pressures for the fastest growing cells. Cells containing recombinant DNA or carrying point mutations leading to the desired overproduction phenotype are susceptible to back-mutation into the original less productive parental strain. Strains can have single gene deletions to develop compensatory mutations that will tend to restore the wild-type growth phenotype. The faster growing cells usually out-compete their more productive counterparts for limiting nutrients, drastically reducing productivity. Batch processes, on the other hand, limit the number of generations available by not reusing cells at the end of each cycle, thus decreasing the probability of the production strain reverting back to its wild-type phenotype. Finally, continuous processes are more difficult to operate long-term due to potential engineering obstacles such as equipment failure and foreign organism contamination. The consequences of such failures also are much more considerable for a continuous process than with a batch culture.

For small-volume production of specialty chemicals and/or proteins, the productivity increases of continuous processes rarely outweigh the risks associated with strain stability and reliability. However, for the production of large-volume, growth-associated products such as 3-hydroxypropionic acid, the increases in productivity for a continuous process can result in significant economic gains when compared to a batch process. Although the engineering obstacles associated with continuous bioprocess operation would always be present, the strain stability concerns can be overcome through metabolic engineering strategies that reroute metabolic pathways to reduce or avoid negative selective pressures and favor production of the target product during the exponential growth phase.

Biochemical synthesis of 3-HP has been established, and several routes can be found in the propanoate metabolism map in the KEGG pathway database shown in FIG. 3 and found at the URL genome.jp/dbget-bin/show_pathway?map00640. However, complete pathways are not present in certain industrial microbes such as E. coli or S. cerevisiae. One useful organism E. coli, is well known in the art and can produce 3-HP by fermentation via lactic acid by recombinantly expressing lactyl-CoA dehydratase and 3-hydroxypropionyl-CoA dehydratase. This strain is described in U.S. Patent Application 20040076982. A more energetically favorable route is the synthesis of 3-HP from malonic semialdehyde, via the intermediate β-alanine. However, the synthesis route for β-alanine is extensive and has the same energetic barrier as the lactic pathway. A further alternative well known in the art is through expression of a 2,3-aminomutase enzyme which converts L-alanine to β-alanine. Expression of this aminomutase creates a pathway from pyruvate to 3-HP in 4 biochemical steps and is described in U.S. Patent Application 20050221466). A 2-step recombinant pathway for creating 3-HP producing microorganisms from glycerol is described in U.S. Pat. No. 6,852,517.

Employing the methods exemplified above and further illustrated in the Examples below, the methods of the invention enable the construction of cells and organisms that obligatory couple the production of a target biochemical product to growth of the cell or organism engineered to harbor the identified genetic alterations. In this regard, metabolic alterations have been identified that obligatorily couple the production of 3-HP to microorganism growth. Microorganism or microbial strains constructed with the identified metabolic alterations produce elevated levels of 3-HP during the exponential growth phase. These strains can be beneficially used for the commercial production of 3-HP in continuous fermentation process without being subjected to the negative selective pressures described previously.

The non-naturally occurring microorganisms of the invention can be employed in the growth-coupled production of 3-HP. Essentially any quantity, including commercial quantities, can be synthesized using the growth-coupled 3-HP producers of the invention. Because the microorganisms of the invention are engineered to obligatorily couple 3-HP to growth continuous or near-continuous growth processes are particularly useful for biosynthetic production of 3-HP. Such continuous and/or near continuous growth processes are described above and exemplified below in the Examples. Continuous and/or near-continuous microorganism growth process also are well known in the art. Briefly, continuous and/or near-continuous growth processes involve maintaining the microorganism in an exponential growth or logarithmic phase. Procedures include using apparatuses such as the Evolugator™ evolution machine (Evolugate LLC, Gainesville, Fla.), fermentors and the like. Additionally, shake flask fermentation and growth under microaerobic conditions also can be employed. Given the teachings and guidance provided herein those skilled in the art will understand that the growth-coupled 3-HP producing microorganisms can be employed in a variety of different settings under a variety of different conditions using a variety of different processes and/or apparatuses well known in the art.

Generally, the continuous and/or near-continuous production of 3-HP will include culturing a non-naturally occurring growth-coupled 3-HP producing organism of the invention in sufficient nutrients and medium to sustain and/or nearly sustain growth in an exponential phase. Continuous culture under such conditions can include, for example, a day, 2, 3, 4, 5, 6 or 7 days or more. Additionally, continuous culture can include 1 week, 2, 3, 4 or 5 or more weeks and up to several months. In is to be understood that the continuous and/or near-continuous culture conditions also can include all time intervals in between these exemplary periods.

3-HP can be harvested or isolated at any time point during the continuous and/or near-continuous culture period exemplified above. As exemplified below in the Examples, the longer the microorganisms are maintained in a continuous and/or near-continuous growth phase, the proportionally greater amount of 3-HP can be produced.

Therefore, the invention provides a method of producing 3-hydroxypropionic acid coupled to the growth of a microorganism. The method includes: (a) culturing a non-naturally occurring microbial organism having at least one exogenous nucleic acid encoding a 3-HP pathway enzyme expressed in a sufficient amount to produce 3-HP under conditions and for a sufficient period of time to produce 3-HP, wherein the 3-HP pathway includes a decarboxylase.

The growth medium can be, for example, any carbohydrate source which can supply a source of carbon to the non-naturally occurring microorganism. Such sources include, for example, sugars such as glucose, xylose, arabinose, galactose, mannose, fructose and starch. Other sources of carbohydrate include, for example, renewable feedstocks and biomass. Exemplary types of biomasses that can be used as feedstocks in the methods of the invention include cellulosic biomass, hemicellulosic biomass and lignin feedstocks or portions of feedstocks. Such biomass feedstocks contain, for example, carbohydrate substrates useful as carbon sources such as glucose, xylose, arabinose, galactose, mannose, fructose and starch. Given the teachings and guidance provided herein, those skilled in the art will understand that renewable feedstocks and biomass other than those exemplified above also can be used for culturing the microbial organisms of the invention for the production of 3-HP.

In addition to renewable feedstocks such as those exemplified above, the 3-HP microbial organisms of the invention also can be modified for growth on syngas as its source of carbon. In this specific embodiment, one or more proteins or enzymes are expressed in the 3-HP producing organisms to provide a metabolic pathway for utilization of syngas or other gaseous carbon source.

Synthesis gas, also known as syngas or producer gas, is the major product of gasification of coal and of carbonaceous materials such as biomass materials, including agricultural crops and residues. Syngas is a mixture primarily of H₂ and CO and can be obtained from the gasification of any organic feedstock, including but not limited to coal, coal oil, natural gas, biomass, and waste organic matter. Gasification is generally carried out under a high fuel to oxygen ratio. Although largely H₂ and CO, syngas can also include CO₂ and other gases in smaller quantities. Thus, synthesis gas provides a cost effective source of gaseous carbon such as CO and, additionally, CO₂.

The Wood-Ljungdahl pathway catalyzes the conversion of CO and H₂ to acetyl-CoA and other products such as acetate. Organisms capable of utilizing CO and syngas also generally have the capability of utilizing CO₂ and CO₂/H₂ mixtures through the same basic set of enzymes and transformations encompassed by the Wood-Ljungdahl pathway. H₂-dependent conversion of CO₂ to acetate by microorganisms was recognized long before it was revealed that CO also could be used by the same organisms and that the same pathways were involved. Many acetogens have been shown to grow in the presence of CO₂ and produce compounds such as acetate as long as hydrogen is present to supply the necessary reducing equivalents (see for example, Drake, Acetogenesis, pp. 3-60 Chapman and Hall, New York, (1994)). This can be summarized by the following equation:

2CO₂+4H₂+n ADP+n Pi→CH₃COOH+2H₂O+n ATP

Hence, non-naturally occurring microorganisms possessing the Wood-Ljungdahl pathway can utilize CO₂ and H₂ mixtures as well for the production of acetyl-CoA and other desired products.

The Wood-Ljungdahl pathway is well known in the art and consists of 12 reactions which can be separated into two branches: (1) methyl branch and (2) carbonyl branch. The methyl branch converts syngas to methyl-tetrahydrofolate (methyl-THF) whereas the carbonyl branch converts methyl-THF to acetyl-CoA. The reactions in the methyl branch are catalyzed in order by the following enzymes or proteins: ferredoxin oxidoreductase, formate dehydrogenase, formyltetrahydrofolate synthetase, methenyltetrahydrofolate cyclodehydratase, methylenetetrahydrofolate dehydrogenase and methylenetetrahydrofolate reductase. The reactions in the carbonyl branch are catalyzed in order by the following enzymes or proteins: cobalamide corrinoid/iron-sulfur protein, methyltransferase, carbon monoxide dehydrogenase, acetyl-CoA synthase, acetyl-CoA synthase disulfide reductase and hydrogenase. Following the teachings and guidance provided herein for introducing a sufficient number of encoding nucleic acids to generate a 3-HP pathway, those skilled in the art will understand that the same engineering design also can be performed with respect to introducing at least the nucleic acids encoding the Wood-Ljungdahl enzymes or proteins absent in the host organism. Therefore, introduction of one or more encoding nucleic acids into the microbial organisms of the invention such that the modified organism contains the complete Wood-Ljungdahl pathway will confer syngas utilization ability.

Accordingly, given the teachings and guidance provided herein, those skilled in the art will understand that a non-naturally occurring microbial organism can be produced that secretes the biosynthesized compounds of the invention when grown on a carbon source such as a carbohydrate. Such compounds include, for example, 3-HP and any of the intermediate metabolites in the 3-HP pathway. All that is required is to engineer in one or more of the required enzyme or protein activities to achieve biosynthesis of the desired compound or intermediate including, for example, inclusion of some or all of the 3-HP biosynthetic pathways. Accordingly, the invention provides a non-naturally occurring microbial organism that produces and/or secretes 3-HP when grown on a carbohydrate or other carbon source and produces and/or secretes any of the intermediate metabolites shown in the 3-HP pathway when grown on a carbohydrate or other carbon source. The 3-HP producing microbial organisms of the invention can initiate synthesis from an intermediate, for example, oxaloacetate and/or malonate semialdehyde.

The non-naturally occurring microbial organisms of the invention are constructed using methods well known in the art as exemplified herein to exogenously express at least one nucleic acid encoding a 3-HP pathway enzyme or protein in sufficient amounts to produce 3-HP. It is understood that the microbial organisms of the invention are cultured under conditions sufficient to produce 3-HP. Following the teachings and guidance provided herein, the non-naturally occurring microbial organisms of the invention can achieve biosynthesis of 3-HP resulting in intracellular concentrations between about 0.1-200 mM or more. Generally, the intracellular concentration of 3-HP is between about 3-150 mM, particularly between about 5-125 mM and more particularly between about 8-100 mM, including about 10 mM, 20 mM, 50 mM, 80 mM, or more. In some embodiments, it is desirable to effect concentrations of 3-HP on the order of 1,000-1,500 mM. Intracellular concentrations between and above each of these exemplary ranges also can be achieved from the non-naturally occurring microbial organisms of the invention.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. patent application Ser. No. 11/891,602, filed Aug. 10, 2007. Any of these conditions can be employed with the non-naturally occurring microbial organisms as well as other anaerobic conditions well known in the art. Under such anaerobic conditions, the 3-HP producers can synthesize 3-HP at intracellular concentrations of 5-10 mM or more as well as all other concentrations exemplified herein. It is understood that, even though the above description refers to intracellular concentrations, 3-HP producing microbial organisms can produce 3-HP intracellularly and/or secrete the product into the culture medium.

The culture conditions can include, for example, liquid culture procedures as well as fermentation and other large scale culture procedures. As described herein, particularly useful yields of the biosynthetic products of the invention can be obtained under anaerobic or substantially anaerobic culture conditions.

As described herein, one exemplary growth condition for achieving biosynthesis of 3-HP includes anaerobic culture or fermentation conditions. In certain embodiments, the non-naturally occurring microbial organisms of the invention can be sustained, cultured or fermented under anaerobic or substantially anaerobic conditions. Briefly, anaerobic conditions refers to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

The culture conditions described herein can be scaled up and grown continuously for manufacturing of 3-HP. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of 3-HP. Generally, and as with non-continuous culture procedures, the continuous and/or near-continuous production of 3-HP will include culturing a non-naturally occurring 3-HP producing organism of the invention in sufficient nutrients and medium to sustain and/or nearly sustain growth in an exponential phase. Continuous culture under such conditions can include, for example, 1 day, 2, 3, 4, 5, 6 or 7 days or more. Additionally, continuous culture can include 1 week, 2, 3, 4 or 5 or more weeks and up to several months. Alternatively, organisms of the invention can be cultured for hours, if suitable for a particular application. It is to be understood that the continuous and/or near-continuous culture conditions also can include all time intervals in between these exemplary periods. It is further understood that the time of culturing the microbial organism of the invention is for a sufficient period of time to produce a sufficient amount of product for a desired purpose.

Fermentation procedures are well known in the art. Briefly, fermentation for the biosynthetic production of 3-HP can be utilized in, for example, fed-batch fermentation and batch separation; fed-batch fermentation and continuous separation, or continuous fermentation and continuous separation. Examples of batch and continuous fermentation procedures are well known in the art.

In addition to the above fermentation procedures using the 3-HP producers of the invention for continuous production of substantial quantities of 3-HP, the 3-HP producers also can be, for example, simultaneously subjected to chemical synthesis procedures to convert the product to other compounds or the product can be separated from the fermentation culture and sequentially subjected to chemical conversion to convert the product to other compounds, if desired.

Directed evolution is a powerful approach that involves the introduction of mutations targeted to a specific gene in order to improve and/or alter the properties of an enzyme. Improved and/or altered enzymes can be identified through the development and implementation of sensitive high-throughput screening assays that allow the automated screening of many enzyme variants (e.g., >10⁴). Iterative rounds of mutagenesis and screening typically are performed to afford an enzyme with optimized properties. Computational algorithms that can help to identify areas of the gene for mutagenesis also have been developed and can significantly reduce the number of enzyme variants that need to be generated and screened.

Numerous directed evolution technologies have been developed (for reviews, see Hibbert et al., Biomol. Eng 22:11-19 (2005); Huisman and Lalonde, In Biocatalysis in the pharmaceutical and biotechnology industries pgs. 717-742 (2007); Patel (ed.), CRC Press; Otten and Quax. Biomol. Eng 22:1-9 (2005).; and Sen et al., Appl Biochem. Biotechnol 143:212-223 (2007)) to be effective at creating diverse variant libraries and these methods have been successfully applied to the improvement of a wide range of properties across many enzyme classes.

Enzyme characteristics that have been improved and/or altered by directed evolution technologies include, for example, selectivity/specificity—for conversion of non-natural substrates; temperature stability—for robust high temperature processing; pH stability—for bioprocessing under lower or higher pH conditions; substrate or product tolerance—so that high product titers can be achieved; binding (Kₘ)—broadens substrate binding to include non-natural substrates; inhibition (Kᵢ)—to remove inhibition by products, substrates, or key intermediates; activity (kcat)—increases enzymatic reaction rates to achieve desired flux; expression levels—increases protein yields and overall pathway flux; oxygen stability—for operation of air sensitive enzymes under aerobic conditions; and anaerobic activity—for operation of an aerobic enzyme in the absence of oxygen.

The following exemplary methods have been developed for the mutagenesis and diversification of genes to target desired properties of specific enzymes. Any of these can be used to alter/optimize activity of a decarboxylase enzyme.

EpPCR (Pritchard et al., J. Theor. Biol. 234:497-509 (2005)) introduces random point mutations by reducing the fidelity of DNA polymerase in PCR reactions by the addition of Mn²⁺ ions, by biasing dNTP concentrations, or by other conditional variations. The five step cloning process to confine the mutagenesis to the target gene of interest involves: 1) error-prone PCR amplification of the gene of interest; 2) restriction enzyme digestion; 3) gel purification of the desired DNA fragment; 4) ligation into a vector; 5) transformation of the gene variants into a suitable host and screening of the library for improved performance. This method can generate multiple mutations in a single gene simultaneously, which can be useful. A high number of mutants can be generated by EpPCR, so a high-throughput screening assay or a selection method (especially using robotics) is useful to identify those with desirable characteristics.

Error-prone Rolling Circle Amplification (epRCA) (Fujii et al., Nucleic Acids Res 32:e145 (2004); and Fujii et al., Nat. Protoc. 1:2493-2497 (2006)) has many of the same elements as epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats. Adjusting the Mn²⁺ concentration can vary the mutation rate somewhat. This technique uses a simple error-prone, single-step method to create a full copy of the plasmid with 3-4 mutations/kbp. No restriction enzyme digestion or specific primers are required. Additionally, this method is typically available as a kit.

DNA or Family Shuffling (Stemmer, Proc Natl Acad Sci U.S.A. 91:10747-10751 (1994); and Stemmer, Nature 370:389-391 (1994)) typically involves digestion of two or more variant genes with nucleases such as Dnase I or EndoV to generate a pool of random fragments that are reassembled by cycles of annealing and extension in the presence of DNA polymerase to create a library of chimeric genes. Fragments prime each other and recombination occurs when one copy primes another copy (template switch). This method can be used with >1 kbp DNA sequences. In addition to mutational recombinants created by fragment reassembly, this method introduces point mutations in the extension steps at a rate similar to error-prone PCR. The method can be used to remove deleterious, random and neutral mutations that might confer antigenicity.

Staggered Extension (StEP) (Zhao et al., Nat. Biotechnol 16:258-261 (1998)) entails template priming followed by repeated cycles of 2 step PCR with denaturation and very short duration of annealing/extension (as short as 5 sec). Growing fragments anneal to different templates and extend further, which is repeated until full-length sequences are made. Template switching means most resulting fragments have multiple parents. Combinations of low-fidelity polymerases (Taq and Mutazyme) reduce error-prone biases because of opposite mutational spectra.

In Random Priming Recombination (RPR) random sequence primers are used to generate many short DNA fragments complementary to different segments of the template. (Shao et al., Nucleic Acids Res 26:681-683 (1998)) Base misincorporation and mispriming via epPCR give point mutations. Short DNA fragments prime one another based on homology and are recombined and reassembled into full-length by repeated thermocycling. Removal of templates prior to this step assures low parental recombinants. This method, like most others, can be performed over multiple iterations to evolve distinct properties. This technology avoids sequence bias, is independent of gene length, and requires very little parent DNA for the application.

In Heteroduplex Recombination linearized plasmid DNA is used to form heteroduplexes that are repaired by mismatch repair. (Volkov et al, Nucleic Acids Res 27:e18 (1999); and Volkov et al., Methods Enzymol. 328:456-463 (2000)) The mismatch repair step is at least somewhat mutagenic. Heteroduplexes transform more efficiently than linear homoduplexes. This method is suitable for large genes and whole operons.

Random Chimeragenesis on Transient Templates (RACHITT) (Coco et al., Nat. Biotechnol 19:354-359 (2001)) employs Dnase I fragmentation and size fractionation of ssDNA. Homologous fragments are hybridized in the absence of polymerase to a complementary ssDNA scaffold. Any overlapping unhybridized fragment ends are trimmed down by an exonuclease. Gaps between fragments are filled in, and then ligated to give a pool of full-length diverse strands hybridized to the scaffold (that contains U to preclude amplification). The scaffold then is destroyed and is replaced by a new strand complementary to the diverse strand by PCR amplification. The method involves one strand (scaffold) that is from only one parent while the priming fragments derive from other genes; the parent scaffold is selected against. Thus, no reannealing with parental fragments occurs. Overlapping fragments are trimmed with an exonuclease. Otherwise, this is conceptually similar to DNA shuffling and StEP. Therefore, there should be no siblings, few inactives, and no unshuffled parentals. This technique has advantages in that few or no parental genes are created and many more crossovers can result relative to standard DNA shuffling.

Recombined Extension on Truncated templates (RETT) entails template switching of unidirectionally growing strands from primers in the presence of unidirectional ssDNA fragments used as a pool of templates. (Lee et al., J. Molec. Catalysis 26:119-129 (2003)) No DNA endonucleases are used. Unidirectional ssDNA is made by DNA polymerase with random primers or serial deletion with exonuclease. Unidirectional ssDNA are only templates and not primers. Random priming and exonucleases don't introduce sequence bias as true of enzymatic cleavage of DNA shuffling/RACHITT. RETT can be easier to optimize than StEP because it uses normal PCR conditions instead of very short extensions. Recombination occurs as a component of the PCR steps—no direct shuffling. This method can also be more random than StEP due to the absence of pauses.

In Degenerate Oligonucleotide Gene Shuffling (DOGS) degenerate primers are used to control recombination between molecules; (Bergquist and Gibbs, Methods Mol. Biol 352:191-204 (2007); Bergquist et al., Biomol. Eng 22:63-72 (2005); Gibbs et al., Gene 271:13-20 (2001)) this can be used to control the tendency of other methods such as DNA shuffling to regenerate parental genes. This method can be combined with random mutagenesis (epPCR) of selected gene segments. This can be a good method to block the reformation of parental sequences. No endonucleases are needed. By adjusting input concentrations of segments made, one can bias towards a desired backbone. This method allows DNA shuffling from unrelated parents without restriction enzyme digests and allows a choice of random mutagenesis methods.

Incremental Truncation for the Creation of Hybrid Enzymes (ITCHY) creates a combinatorial library with 1 base pair deletions of a gene or gene fragment of interest. (Ostermeier et al., Proc Natl Acad Sci U.S.A. 96:3562-3567 (1999); and Ostermeier et al., Nat. Biotechnol 17:1205-1209 (1999)) Truncations are introduced in opposite direction on pieces of 2 different genes. These are ligated together and the fusions are cloned. This technique does not require homology between the 2 parental genes. When ITCHY is combined with DNA shuffling, the system is called SCRATCHY (see below). A major advantage of both is no need for homology between parental genes; for example, functional fusions between an E. coli and a human gene were created via ITCHY. When ITCHY libraries are made, all possible crossovers are captured.

Thio-Incremental Truncation for the Creation of Hybrid Enzymes (THIO-ITCHY) is similar to ITCHY except that phosphothioate dNTPs are used to generate truncations. (Lutz et al., Nucleic Acids Res 29:E16 (2001)) Relative to ITCHY, THIO-ITCHY can be easier to optimize, provide more reproducibility, and adjustability.

SCRATCHY combines two methods for recombining genes, ITCHY and DNA shuffling. (Lutz et al., Proc Natl Acad Sci U.S.A. 98:11248-11253 (2001)) SCRATCHY combines the best features of ITCHY and DNA shuffling. First, ITCHY is used to create a comprehensive set of fusions between fragments of genes in a DNA homology-independent fashion. This artificial family is then subjected to a DNA-shuffling step to augment the number of crossovers. Computational predictions can be used in optimization. SCRATCHY is more effective than DNA shuffling when sequence identity is below 80%.

In Random Drift Mutagenesis (RNDM) mutations made via epPCR followed by screening/selection for those retaining usable activity. (Bergquist et al., Biomol. Eng 22:63-72 (2005)) Then, these are used in DOGS to generate recombinants with fusions between multiple active mutants or between active mutants and some other desirable parent. Designed to promote isolation of neutral mutations; its purpose is to screen for retained catalytic activity whether or not this activity is higher or lower than in the original gene. RNDM is usable in high throughput assays when screening is capable of detecting activity above background. RNDM has been used as a front end to DOGS in generating diversity. The technique imposes a requirement for activity prior to shuffling or other subsequent steps; neutral drift libraries are indicated to result in higher/quicker improvements in activity from smaller libraries. Though published using epPCR, this could be applied to other large-scale mutagenesis methods.

Sequence Saturation Mutagenesis (SeSaM) is a random mutagenesis method that: 1) generates pool of random length fragments using random incorporation of a phosphothioate nucleotide and cleavage; this pool is used as a template to 2) extend in the presence of “universal” bases such as inosine; 3) replication of a inosine-containing complement gives random base incorporation and, consequently, mutagenesis. (Wong et al., Biotechnol J 3:74-82 (2008); Wong et al., Nucleic Acids Res 32:e26 (2004); and Wong et al., Anal. Biochem 341:187-189 (2005)) Using this technique it can be possible to generate a large library of mutants within 2-3 days using simple methods. This technique is non-directed in comparison to the mutational bias of DNA polymerases. Differences in this approach makes this technique complementary (or an alternative) to epPCR.

In Synthetic Shuffling, overlapping oligonucleotides are designed to encode “all genetic diversity in targets” and allow a very high diversity for the shuffled progeny. (Ness et al., Nat. Biotechnol 20:1251-1255 (2002)) In this technique, one can design the fragments to be shuffled. This aids in increasing the resulting diversity of the progeny. One can design sequence/codon biases to make more distantly related sequences recombine at rates approaching those observed with more closely related sequences. Additionally, the technique does not require physically possessing the template genes.

Nucleotide Exchange and Excision Technology NexT exploits a combination of dUTP incorporation followed by treatment with uracil DNA glycosylase and then piperidine to perform endpoint DNA fragmentation. (Muller et al., Nucleic Acids Res 33:e117 (2005)) The gene is reassembled using internal PCR primer extension with proofreading polymerase. The sizes for shuffling are directly controllable using varying dUPT::dTTP ratios. This is an end point reaction using simple methods for uracil incorporation and cleavage. Other nucleotide analogs, such as 8-oxo-guanine, can be used with this method. Additionally, the technique works well with very short fragments (86 bp) and has a low error rate. The chemical cleavage of DNA used in this technique results in very few unshuffled clones.

In Sequence Homology-Independent Protein Recombination (SHIPREC) a linker is used to facilitate fusion between two distantly/unrelated genes. Nuclease treatment is used to generate a range of chimeras between the two genes. These fusions result in libraries of single-crossover hybrids. (Sieber et al., Nat. Biotechnol 19:456-460 (2001)) This produces a limited type of shuffling and a separate process is required for mutagenesis. In addition, since no homology is needed this technique can create a library of chimeras with varying fractions of each of the two unrelated parent genes. SHIPREC was tested with a heme-binding domain of a bacterial CP450 fused to N-terminal regions of a mammalian CP450; this produced mammalian activity in a more soluble enzyme.

In Gene Site Saturation Mutagenesis™ (GSSM™) the starting materials are a supercoiled dsDNA plasmid containing an insert and two primers which are degenerate at the desired site of mutations. (Kretz et al., Methods Enzymol. 388:3-11 (2004)) Primers carrying the mutation of interest, anneal to the same sequence on opposite strands of DNA. The mutation is typically in the middle of the primer and flanked on each side by ˜20 nucleotides of correct sequence. The sequence in the primer is NNN or NNK (coding) and MNN (noncoding) (N=all 4, K=G, T, M=A, C). After extension, DpnI is used to digest dam-methylated DNA to eliminate the wild-type template. This technique explores all possible amino acid substitutions at a given locus (i.e., one codon). The technique facilitates the generation of all possible replacements at a single-site with no nonsense codons and results in equal to near-equal representation of most possible alleles. This technique does not require prior knowledge of the structure, mechanism, or domains of the target enzyme. If followed by shuffling or Gene Reassembly, this technology creates a diverse library of recombinants containing all possible combinations of single-site up-mutations. The utility of this technology combination has been demonstrated for the successful evolution of over 50 different enzymes, and also for more than one property in a given enzyme.

Combinatorial Cassette Mutagenesis (CCM) involves the use of short oligonucleotide cassettes to replace limited regions with a large number of possible amino acid sequence alterations. (Reidhaar-Olson et al. Methods Enzymol. 208:564-586 (1991); and Reidhaar-Olson et al. Science 241:53-57 (1988)) Simultaneous substitutions at two or three sites are possible using this technique. Additionally, the method tests a large multiplicity of possible sequence changes at a limited range of sites. This technique has been used to explore the information content of the lambda repressor DNA-binding domain.

Combinatorial Multiple Cassette Mutagenesis (CMCM) is essentially similar to CCM except it is employed as part of a larger program: 1) Use of epPCR at high mutation rate to 2) ID hot spots and hot regions and then 3) extension by CMCM to cover a defined region of protein sequence space. (Reetz et al., Angew. Chem. Int. Ed Engl. 40:3589-3591 (2001)) As with CCM, this method can test virtually all possible alterations over a target region. If used along with methods to create random mutations and shuffled genes, it provides an excellent means of generating diverse, shuffled proteins. This approach was successful in increasing, by 51-fold, the enantioselectivity of an enzyme.

In the Mutator Strains technique conditional ts mutator plasmids allow increases of 20- to 4000-X in random and natural mutation frequency during selection and block accumulation of deleterious mutations when selection is not required. (Selifonova et al., Appl Environ Microbiol 67:3645-3649 (2001)) This technology is based on a plasmid-derived mutD5 gene, which encodes a mutant subunit of DNA polymerase III. This subunit binds to endogenous DNA polymerase III and compromises the proofreading ability of polymerase III in any strain that harbors the plasmid. A broad-spectrum of base substitutions and frameshift mutations occur. In order for effective use, the mutator plasmid should be removed once the desired phenotype is achieved; this is accomplished through a temperature sensitive origin of replication, which allows for plasmid curing at 41° C. It should be noted that mutator strains have been explored for quite some time (e.g., see Low et al., J. Mol. Biol. 260:359-3680 (1996)). In this technique very high spontaneous mutation rates are observed. The conditional property minimizes non-desired background mutations. This technology could be combined with adaptive evolution to enhance mutagenesis rates and more rapidly achieve desired phenotypes.

“Look-Through Mutagenesis (LTM) is a multidimensional mutagenesis method that assesses and optimizes combinatorial mutations of selected amino acids.” (Rajpal et al., Proc Natl Acad Sci U.S.A. 102:8466-8471 (2005)) Rather than saturating each site with all possible amino acid changes, a set of nine is chosen to cover the range of amino acid R-group chemistry. Fewer changes per site allows multiple sites to be subjected to this type of mutagenesis. A >800-fold increase in binding affinity for an antibody from low nanomolar to picomolar has been achieved through this method. This is a rational approach to minimize the number of random combinations and can increase the ability to find improved traits by greatly decreasing the numbers of clones to be screened. This has been applied to antibody engineering, specifically to increase the binding affinity and/or reduce dissociation. The technique can be combined with either screens or selections.

Gene Reassembly is a DNA shuffling method that can be applied to multiple genes at one time or to creating a large library of chimeras (multiple mutations) of a single gene. (Tunable GeneReassembly™ (TGR™) Technology supplied by Verenium Corporation) Typically this technology is used in combination with ultra-high-throughput screening to query the represented sequence space for desired improvements. This technique allows multiple gene recombination independent of homology. The exact number and position of cross-over events can be pre-determined using fragments designed via bioinformatic analysis. This technology leads to a very high level of diversity with virtually no parental gene reformation and a low level of inactive genes. Combined with GSSM™, a large range of mutations can be tested for improved activity. The method allows “blending” and “fine tuning” of DNA shuffling, e.g. codon usage can be optimized.

In Silico Protein Design Automation (PDA) is an optimization algorithm that anchors the structurally defined protein backbone possessing a particular fold, and searches sequence space for amino acid substitutions that can stabilize the fold and overall protein energetics. (Hayes et al., Proc Natl Acad Sci U.S.A. 99:15926-15931 (2002)) This technology uses in silico structure-based entropy predictions in order to search for structural tolerance toward protein amino acid variations. Statistical mechanics is applied to calculate coupling interactions at each position. Structural tolerance toward amino acid substitution is a measure of coupling. Ultimately, this technology is designed to yield desired modifications of protein properties while maintaining the integrity of structural characteristics. The method computationally assesses and allows filtering of a very large number of possible sequence variants (10⁵⁰). The choice of sequence variants to test is related to predictions based on the most favorable thermodynamics. Ostensibly only stability or properties that are linked to stability can be effectively addressed with this technology. The method has been successfully used in some therapeutic proteins, especially in engineering immunoglobulins. In silico predictions avoid testing extraordinarily large numbers of potential variants. Predictions based on existing three-dimensional structures are more likely to succeed than predictions based on hypothetical structures. This technology can readily predict and allow targeted screening of multiple simultaneous mutations, something not possible with purely experimental technologies due to exponential increases in numbers.

Iterative Saturation Mutagenesis (ISM) involves: 1) use knowledge of structure/function to choose a likely site for enzyme improvement; 2) saturation mutagenesis at chosen site using Stratagene QuikChange (or other suitable means); 3) screen/select for desired properties; and 4) with improved clone(s), start over at another site and continue repeating. (Reetz et al., Nat. Protoc. 2:891-903 (2007); and Reetz et al., Angew. Chem. Int. Ed Engl. 45:7745-7751 (2006)) This is a proven methodology, which assures all possible replacements at a given position are made for screening/selection.

Any of the aforementioned methods for mutagenesis can be used alone or in any combination. Additionally, any one or combination of the directed evolution methods can be used in conjunction with adaptive evolution techniques.

Adaptive evolution is a powerful experimental technique that can be used to increase growth rates of mutant or engineered microbial strains, or of wild-type strains growing under unnatural environmental conditions. It is especially useful for strains designed via the OptKnock formalism, which results in growth-coupled product formation. Therefore, evolution toward optimal growing strains will indirectly optimize production as well. Unique strains of E. coli K-12 MG1655 were created through gene knockouts and adaptive evolution. (Fong and Palsson, Nat. Genet. 36:1056-1058 (2004)) In this work, all adaptive evolutionary cultures were maintained in prolonged exponential growth by serial passage of batch cultures into fresh medium before the stationary phase was reached, thus rendering growth rate as the primary selection pressure. The genes that were selected for this knockout study were ackA, frdA, pckA, ppc, tpiA, and zwf. Knockout strains were constructed and evolved on minimal medium supplemented with different carbon substrates (four for each knockout strain). Evolution cultures were carried out in duplicate or triplicate, giving a total of 50 evolution knockout strains. The evolution cultures were maintained in exponential growth until a stable growth rate was reached. The computational predictions were accurate (i.e., within 10%) at predicting the post-evolution growth rate of the knockout strains in 38 out of the 50 cases examined. Furthermore, a combination of OptKnock design with adaptive evolution has led to improved lactic acid production strains. (Fong et al., Biotechnol Bioeng 91:643-648 (2005).)

There are a number of developed technologies for carrying out adaptive evolution. Exemplary methods are provided herein below. In some embodiments, optimization of a non-naturally occurring organism of the present invention includes subject the use of any of the these adaptive evolution techniques.

Serial culture involves repetitive transfer of a small volume of grown culture to a much larger vessel containing fresh growth medium. When the cultured organisms have grown to saturation in the new vessel, the process is repeated. This method has been used to achieve the longest demonstrations of sustained culture in the literature, (Lenski and Travisano, Proc. Natl. Acad. Sci. U.S.A. 91:6808-6814 (1994)) in experiments which clearly demonstrated consistent improvement in reproductive rate over period of years. In the experiments performed in the Palsson lab described above, transfer is usually performed during exponential phase, so each day the transfer volume is precisely calculated to maintain exponential growth through the next 24 hour period. This process is usually done manually, with considerable labor investment, and is subject to contamination through exposure to the outside environment. Furthermore, since such small volumes are transferred each time, the evolution is inefficient and many beneficial mutations are lost. On the positive side, serial dilution is inexpensive and easy to parallelize.

In continuous culture the growth of cells in a chemostat represents an extreme case of dilution in which a very high fraction of the cell population remains. As a culture grows and becomes saturated, a small proportion of the grown culture is replaced with fresh media, allowing the culture to continually grow at close to its maximum population size. Chemostats have been used to demonstrate short periods of rapid improvement in reproductive rate. (Dykhuizen, D. E., Methods Enzymol. 613-631 (1993).) The potential power of these devices was recognized, but traditional chemostats were unable to sustain long periods of selection for increased reproduction rate, due to the unintended selection of dilution-resistant (static) variants. These variants are able to resist dilution by adhering to the surface of the chemostat, and by doing so, outcompete less sticky individuals including those that have higher reproductive rates, thus obviating the intended purpose of the device. (Chao and Ramsdell, J. Gen. Microbiol. 20:132-138 (1985).) One way to overcome this drawback is the implementation of a device with two growth chambers, which periodically undergo transient phases of sterilization, as described in the patent by the Pasteur Institute (Marliere and Mutzel, U.S. Pat. No. 6,686,194, filed 1999).

EVOLUGATOR™ is a continuous culture device developed by Evolugate, LLC (Gainesville, Fla.) exhibits significant time and effort savings over traditional evolution techniques. (de Crecy et al., Appl. Microbiol. Biotechnol. 77:489-496 (2007).) The cells are maintained in prolonged exponential growth by the serial passage of batch cultures into fresh medium before the stationary phase is attained. By automating optical density measurement and liquid handling, EVOLUGATOR™ can perform serial transfer at high rates using large culture volumes, thus approaching the efficiency of a chemostat in evolution of cell fitness. For example, a mutant of Acinetobacter sp ADP1 deficient in a component of the translation apparatus, and having severely hampered growth, was evolved in 200 generations to 80% of the wild-type growth rate. However, in contrast to the chemostat which maintains cells in a single vessel, the machine operates by moving from one “reactor” to the next in subdivided regions of a spool of tubing, thus eliminating any selection for wall-growth. The transfer volume is adjustable, and normally set to about 50%. A drawback to this device is that it is large and costly, thus running large numbers of evolutions in parallel is impractical. Furthermore, gas addition is not well regulated, and strict anaerobic conditions are not maintained with the current device configuration.

Using the methods described herein, one skilled in the art will be able to readily isolate and characterize, using standard methods, a nucleic acid sequence encoding a decarboxylase capable of catalyzing the decarboxylation of oxaloacetate to malonate semialdehyde. Once identified standard methods in the art can be used to introduce it into a host microbial organism to create a non-naturally occurring organism having the nucleic acid encoding such a decarboxylase. Likewise, standard techniques in the art can be used to isolate and characterize such decarboxylases. Furthermore, it will be readily apparent that computer modeling of the enzyme active site of the decarboxylase and/or data obtained from crystal structures, nuclear magnetic resonance (NMR) data, or the like, can provide further insight into the structure-function relationships for the decarboxylase. This can be used to advantage for further manipulation of the nucleic acid sequence encoding the decarboxylase or to create post-translational modifications to the decarboxylase.

To generate better producers, metabolic modeling can be utilized to optimize growth conditions to provide gene knockout designs that additionally optimize utilization of the pathways, as described above. Modeling analysis allows reliable predictions of the effects on cell growth of shifting the metabolism towards more efficient production of 3-HP.

It is understood that modifications which do not substantially affect the activity of the various embodiments of this invention are also included within the definition of the invention provided herein. Accordingly, the following examples are intended to illustrate but not limit the present invention.

### Example I

### 3-HP Biosynthesis

This Example describes the generation of a microbial organism capable of producing 3-HP using a 2-keto acid decarboxylase metabolic pathway.

Escherichia coli is used as a target organism to engineer a 3-HP pathway, and testing growth and 3-HP production from glucose. E. coli provides a good model for developing a non-naturally occurring microorganism capable of producing 3-HP, from glucose since it is amenable to genetic manipulation and is known to be capable of producing various products, like ethanol, effectively under anaerobic conditions from glucose.

To generate an E. coli strain engineered to produce 3-HP, nucleic acids encoding proteins and enzymes required for the 3-HP production pathway via oxaloacetate decarboxylation as described above, are expressed in E. coli using well known molecular biology techniques (see, for example, Sambrook, supra, 2001; Ausubel supra, 1999; Roberts et al., supra, 1989). The mdlC gene (P20906.2), encoding a decarboxylase, the pckA (P43923.1) gene, encoding a PPCK, and the mmsB gene (NP_{—}252259.1), encoding a dehydrogenase are cloned into the pZE13 vector under the PA1/lacO promoter. (An alternate set of insertions includes kgd gene (O50463.4), encoding a decarboxylase, the pckA (P43923.1) gene, encoding a PPCK, and the mmsB gene (NP_{—}252259.1), encoding a dehydrogenase.) The plasmid is transformed into E. coli strain MG1655 to express the decarboxylase required for decarboxylation of oxaloacetate to malonate semialdehyde, which is then reduced to 3-HP. The expression of the 2-keto acid decarboxylase pathway genes is corroborated using methods well known in the art for determining polypeptide expression or enzymatic activity, including for example, Northern blots, PCR amplification of mRNA, immunoblotting. Enzymatic activities of the expressed enzymes are confirmed using assays specific for the individually activities. The ability of the engineered E. coli strain to produce 3-HP is confirmed using HPLC, gas chromatography-mass spectrometry (GCMS) or liquid chromatography-mass spectrometry (LCMS).

The engineered production organism containing the 2-keto acid decarboxylase pathway enzymes is grown in a 10 L bioreactor sparged with an N₂/CO₂ mixture, using 5 L broth containing 5 g/L potassium phosphate, 2.5 g/L ammonium chloride, 0.5 g/L magnesium sulfate, and 30 g/L corn steep liquor, and an initial glucose concentration of 20 g/L. As the cells grow and utilize the glucose, additional 70% glucose is fed into the bioreactor at a rate approximately balancing glucose consumption. The temperature of the bioreactor is maintained at 30 degrees C. The pH of the medium is maintained at a pH of 7 by addition of an acid, such as H2SO4. The growth rate is determined by measuring optical density using a spectrophotometer (600 nm), and the glucose uptake rate by monitoring carbon source depletion over time. 3-HP and byproducts such as undesirable alcohols, organic acids, and residual glucose can be quantified by HPLC (Shimadzu) with an HPX-087 column (BioRad), using a refractive index detector for glucose and alcohols, and a UV detector for organic acids, Lin et al., Biotechnol. Bioeng., 775-779 (2005). Growth continues for approximately 24 hours, until 3-HP reaches a concentration of between 10-200 g/L, with the cell density being between 5 and 50 g/L. Upon completion of the cultivation period, the fermenter contents are passed through a cell separation unit (e.g., centrifuge) to remove cells and cell debris, and the fermentation broth and 3-HP is separated from the broth and purified by standard methods for organic acid recovery.

### Example II

### 3-HP Biosynthesis via the Malonyl-CoA to 3-HP Pathway

This Example describes the generation of a microbial organism capable of producing 3-HP using a CoA-dependent oxaloacetate dehydrogenase metabolic pathway.

Escherichia coli is used as a target organism to engineer a 3-HP pathway, and testing growth and 3-HP production from glucose. E. coli is amenable to genetic manipulation and various products, like ethanol, have been produced effectively under anaerobic conditions from glucose.

To generate an E. coli strain engineered to produce 3-HP, nucleic acids encoding proteins and enzymes for the 3-HP production pathway via oxaloacetate dehydrogenation as described above, are expressed in E. coli using well known molecular biology techniques (see, for example, Sambrook, supra, 2001; Ausubel supra, 1999; Roberts et al., supra, 1989). In particular, the sucA (NP_{—}415254.1), sucB (NP_{—}415255.1), and lpd (NP_{—}414658.1) genes encoding the CoA-dependent oxaloacetate dehydrogenase activity are cloned into the pZE13 vector (Expressys, Ruelzheim, Germany) under the PA1/lacO promoter. An alternative set of genes encoding CoA-dependent oxaloacetate dehydrogenase activity includes KGD1 (NP_{—}012141.1), KGD2(NP_{—}010432.1),and LPD1 (NP_{—}116635.1). In addition, pckA (P43923.1), Msed_{—}0709 (YP_{—}001190808.1), and mmsB gene (NPD_{—}252259.1) encoding PEP carboxykinase, CoA-acylating malonate semialdehyde dehydrogenase, and dehydrogenase activities, respectively are cloned into the pZA33 vector (Expressys, Ruelzheim, Germany) under the PA1/lacO promoter. An alternative set of genes includes pckA (YP_{—}089485.1), mcr (NP_{—}378167.1), and mmsB (NP_{—}746775.1). The two sets of plasmids are transformed into E. coli strain MG1655 to express the proteins and enzymes required for 3-HP synthesis via the malonyl-CoA to 3-HP pathway. The expression of the CoA-dependent oxaloacetate dehydrogenase pathway genes required for 3-HP synthesis is corroborated using methods well known in the art for determining polypeptide expression or enzymatic activity, including for example, Northern blots, PCR amplification of mRNA, immunoblotting. Enzymatic activities of the expressed enzymes are confirmed using assays specific for the individually activities. The ability of the engineered E. coli strain to produce 3-HP is confirmed using HPLC, gas chromatography-mass spectrometry (GCMS) or liquid chromatography-mass spectrometry (LCMS).

The engineered production organism containing a decarboxylase enzyme is grown in a 10 L bioreactor sparged with an N₂/CO₂ mixture, using 5 L broth containing 5 g/L potassium phosphate, 2.5 g/L ammonium chloride, 0.5 g/L magnesium sulfate, and 30 g/L corn steep liquor, and an initial glucose concentration of 20 g/L. As the cells grow and utilize the glucose, additional 70% glucose is fed into the bioreactor at a rate approximately balancing glucose consumption. The temperature of the bioreactor is maintained at 30 degrees C. The pH of the medium is maintained at a pH of 7 by addition of an acid, such as H2SO4. The growth rate is determined by measuring optical density using a spectrophotometer (600 nm), and the glucose uptake rate by monitoring carbon source depletion over time. 3-HP and byproducts such as undesirable alcohols, organic acids, and residual glucose can be quantified by HPLC (Shimadzu) with an HPX-087 column (BioRad), using a refractive index detector for glucose and alcohols, and a UV detector for organic acids, Lin et al., Biotechnol. Bioeng. 775-779 (2005). Growth continues for approximately 24 hours, until 3-HP reaches a concentration of between 10-200 g/L, with the cell density being between 5 and 50 g/L. Upon completion of the cultivation period, the fermenter contents are passed through a cell separation unit (e.g., centrifuge) to remove cells and cell debris, and the fermentation broth and 3-HP is separated from the broth and purified by standard methods for organic acid recovery.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

What is claimed is:

1. A non-naturally occurring microbial organism, comprising a microbial organism having a 3-hydroxypropanoic acid (3-HP) pathway comprising at least one exogenous nucleic acid encoding 3-HP pathway enzyme expressed in a sufficient amount to produce 3-HP, said 3-HP pathway comprising an 2-keto acid decarboxylase.

2. The non-naturally occurring microbial organism of claim 1, wherein said microbial organism comprises two exogenous nucleic acids each encoding a 3-HP pathway enzyme.

3. The non-naturally occurring microbial organism of claim 1, wherein said microbial organism comprises three exogenous nucleic acids each encoding a 3-HP pathway enzyme.

4. The non-naturally occurring microbial organism of claim 3, wherein said three exogenous nucleic acids encode a phophoenolpyruvate carboxykinase, a 2-keto acid decarboxylase, and a dehydrogenase.

5. The non-naturally occurring microbial organism of claim 3, wherein said three exogenous nucleic acids encode a phophoenolpyruvate carboxykinase, a 2-keto acid decarboxylase, and a 3-hydroxypropionate dehydrogenase.

6. The non-naturally occurring microbial organism of claim 1, wherein said at least one exogenous nucleic acid is a heterologous nucleic acid.

7. The non-naturally occurring microbial organism of claim 1, wherein said non-naturally occurring microbial organism is in a substantially anaerobic culture medium.

8. The non-naturally occurring microbial organism of claim 1 further comprising an exogenous nucleic acid encoding a phosphoenolpyruvate carboxykinase.

9. The non-naturally occurring microbial organism of claim 8, wherein said non-naturally occurring microbial organism is in a substantially anaerobic culture medium.

10. The non-naturally occurring microbial organism of claim 1 further comprising an exogenous nucleic acid encoding a dehydrogenase capable of converting malonate semialdehyde to 3-HP.

11. The non-naturally occurring microbial organism of claim 1 further comprising an exogenous nucleic acid encoding a 3-hydroxypropionate dehydrogenase capable of converting malonate semialdehyde to 3-HP.

12. A method for producing 3-HP, comprising culturing a non-naturally occurring microbial organism having a 3-HP pathway, said pathway comprising at least one exogenous nucleic acid encoding a 3-HP pathway enzyme expressed in a sufficient amount to produce 3-HP under conditions and for a sufficient period of time to produce 3-HP, said 3-HP pathway comprising an 2-keto acid decarboxylase.

13. The method of claim 12, wherein said microbial organism comprises two exogenous nucleic acids each encoding a 3-HP pathway enzyme.

14. The method of claim 13, wherein said microbial organism comprises three exogenous nucleic acids each encoding a 3-HP pathway enzyme.

15. The method of claim 13, wherein said three exogenous nucleic acids encode a phophoenolpyruvate carboxykinase, an 2-keto acid decarboxylase, and a dehydrogenase.

16. The method of claim 13, wherein said three exogenous nucleic acids encode a phophoenolpyruvate carboxykinase, a 2-keto acid decarboxylase, and a 3-hydroxypropionate dehydrogenase.

17. The method of claim 12, wherein said at least one exogenous nucleic acid is a heterologous nucleic acid.

18. The method of claim 12, wherein said non-naturally occurring microbial organism is in a substantially anaerobic culture medium.

19. The method of claim 12, wherein said non-naturally occurring microbial organism further comprises an exogenous nucleic acid encoding a phosphoenolpyruvate carboxykinase.

20. The method of claim 12, wherein said non-naturally occurring microbial organism further comprises an exogenous nucleic acid encoding a dehydrogenase capable of converting malonate semialdehyde to 3-HP.

21. The method of claim 12, wherein said non-naturally occurring microbial organism further comprises an exogenous nucleic acid encoding a 3-hydroxypropionate dehydrogenase capable of converting malonate semialdehyde to 3-HP.

22. A non-naturally occurring microbial organism, comprising a microbial organism having a 3-hydroxypropanoic acid (3-HP) pathway comprising at least one exogenous nucleic acid encoding a 3-HP pathway enzyme expressed in a sufficient amount to produce 3-HP, said 3-HP pathway comprising a CoA-dependent oxaloacetate dehydrogenase.

23. The non-naturally occurring microbial organism of claim 22, wherein said microbial organism comprises two exogenous nucleic acids.

24. The non-naturally occurring microbial organism of claim 22, wherein said microbial organism comprises three exogenous nucleic acids.

25. The non-naturally occurring microbial organism of claim 22, wherein said microbial organism comprises four exogenous nucleic acids.

26. The non-naturally occurring microbial organism of claim 24, wherein said three exogenous nucleic acids encode a phophoenolpyruvate carboxykinase, a CoA-dependent oxaloacetate dehydrogenase, and a malonyl-CoA reductase.

27. The non-naturally occurring microbial organism of claim 25, wherein said four exogenous nucleic acids encode a phophoenolpyruvate carboxykinase, a CoA-dependent oxaloacetate dehydrogenase, a CoA-acylating aldehyde dehydrogenase, and a primary alcohol dehydrogenase.

28. The non-naturally occurring microbial organism of claim 25, wherein said four exogenous nucleic acids encode a phophoenolpyruvate carboxykinase, a CoA-dependent oxaloacetate dehydrogenase, a CoA-acylating malonate semialdehyde dehydrogenase, and a 3-hydroxypropionate dehydrogenase.

29. The non-naturally occurring microbial organism of claim 22, wherein said at least one exogenous nucleic acid is a heterologous nucleic acid.

30. The non-naturally occurring microbial organism of claim 22, wherein said non-naturally occurring microbial organism is in a substantially anaerobic culture medium.

31. The non-naturally occurring microbial organism of claim 22, further comprising a nucleic acid encoding a phosphoenolpyruvate carboxykinase.

32. A method for producing 3-HP, comprising culturing a non-naturally occurring microbial organism having a malonyl-CoA to 3-HP pathway, said pathway comprising at least one exogenous nucleic acid encoding a malonyl-CoA to 3-HP pathway enzyme expressed in a sufficient amount to produce 3-HP under conditions and for a sufficient period of time to produce 3-HP, said malonyl-CoA to 3-HP pathway comprising a CoA-dependent oxaloacetate dehydrogenase.

33. The method of claim 32, wherein said microbial organism comprises two exogenous nucleic acids.

34. The method of claim 32, wherein said microbial organism comprises three exogenous nucleic acids.

35. The method of claim 32, wherein said microbial organism comprises four exogenous nucleic acids.

36. The method of claim 34, wherein said three exogenous nucleic acids encode a phophoenolpyruvate carboxykinase, a CoA-dependent oxaloacetate dehydrogenase, and a malonyl-CoA reductase.

37. The method of claim 35, wherein said four exogenous nucleic acids encode a phophoenolpyruvate carboxykinase, a CoA-dependent oxaloacetate dehydrogenase, a CoA-acylating aldehyde dehydrogenase, and a primary alcohol dehydrogenase.

38. The method of claim 35, wherein said four exogenous nucleic acids encode a phophoenolpyruvate carboxykinase, a CoA-dependent oxaloacetate dehydrogenase, a CoA-acylating malonate semialdehyde dehydrogenase, and a 3-hydroxypropionate dehydrogenase.

39. The method of claim 32, wherein said at least one exogenous nucleic acid is a heterologous nucleic acid.

40. The method of claim 32, wherein said non-naturally occurring microbial organism is in a substantially anaerobic culture medium.

41. The method of claim 32, further comprising an exogenous nucleic acid encoding a phosphoenolpyruvate carboxykinase.

42. A non-naturally occurring microbial organism, comprising a microbial organism having a 3-hydroxypropanoic acid (3-HP) pathway comprising at least one exogenous nucleic acid encoding a 3-HP pathway enzyme expressed in a sufficient amount to produce 3-HP, said 3-HP pathway comprising a malate decarboxylase.

43. The non-naturally occurring microbial organism of claim 42, wherein said microbial organism comprises two exogenous nucleic acids each encoding a 3-HP pathway enzyme.

44. The non-naturally occurring microbial organism of claim 42, wherein said microbial organism comprises three exogenous nucleic acids each encoding a 3-HP pathway enzyme.

45. The non-naturally occurring microbial organism of claim 44, wherein said three exogenous nucleic acids encode a phophoenolpyruvate carboxykinase, a malate dehydrogenase, and a malate decarboxylase.

46. The non-naturally occurring microbial organism of claim 42, wherein said at least one exogenous nucleic acid is a heterologous nucleic acid.

47. The non-naturally occurring microbial organism of claim 42, wherein said non-naturally occurring microbial organism is in a substantially anaerobic culture medium.

48. The non-naturally occurring microbial organism of claim 42 further comprising an exogenous nucleic acid encoding a phosphoenolpyruvate carboxykinase.

49. The non-naturally occurring microbial organism of claim 48, wherein said non-naturally occurring microbial organism is in a substantially anaerobic culture medium.

50. The non-naturally occurring microbial organism of claim 42 further comprising an exogenous nucleic acid encoding a malate dehydrogenase capable of converting oxaloacetate to malate.

51. A method for producing 3-HP, comprising culturing a non-naturally occurring microbial organism having a malonyl-CoA to 3-HP pathway, said pathway comprising at least one exogenous nucleic acid encoding a malate to 3-HP pathway enzyme expressed in a sufficient amount to produce 3-HP under conditions and for a sufficient period of time to produce 3-HP, said malonyl-CoA to 3-HP pathway comprising a malate decarboxylase.

52. The method of claim 51, wherein said microbial organism comprises two exogenous nucleic acids each encoding a 3-HP pathway enzyme.

53. The method of claim 51, wherein said microbial organism comprises three exogenous nucleic acids each encoding a 3-HP pathway enzyme.

54. The method of claim 53, wherein said three exogenous nucleic acids encode a phophoenolpyruvate carboxykinase, a malate dehydrogenase, and a malate decarboxylase.

55. The method of claim 51, wherein said at least one exogenous nucleic acid is a heterologous nucleic acid.

56. The method of claim 51, wherein said non-naturally occurring microbial organism is in a substantially anaerobic culture medium.

57. The method of claim 51 further comprising an exogenous nucleic acid encoding a phosphoenolpyruvate carboxykinase.

58. The method of claim 57, wherein said non-naturally occurring microbial organism is in a substantially anaerobic culture medium.

59. The method of claim 51 further comprising an exogenous nucleic acid encoding a malate dehydrogenase capable of converting oxaloacetate to malate.
